(19) United States
(12) Patent Application Publication
(10) Pub. No.: US 2024/0035966 A1
(43) Pub. Date: Feb. 1, 2024

(54) DEVICE AND METHOD FOR IDENTIFYING PEPTIDES AND PROTEINS IN A FLUID SAMPLE

(71) Applicant: ILOF - INTELLIGENT LAB ON FIBER, LDA, Matosinhos (PT)

(72) Inventors: Cristiana Raquel DA SILVA CARPINTEIRO, Matosinhos (PT); Fillpe Miguel MOREIRA MARQUES, Matosinhos (PT); Joana Isabel SANTOS PAIVA, Matosinhos (PT); Paulo Henrique DA COSTA SANTOS, Matosinhos (PT); Sandra Cristina MARTINS RODRIGUES, Matosinhos (PT); Vanessa Patricia DIAS PINTO, Matosinhos (PT)

(21) Appl. No.: 18/022,187

(22) PCT Filed: Aug. 20, 2021
(86) PCT No.: PCT/EP2021/073187

§ 371 Date: Feb. 20, 2023

(30) Foreign Application Priority Data
Aug. 20, 2020 (LU) LU102007

(51) Int. Cl. G01N 21/47; G16B 50/30

(52) U.S. Cl. CPC G01N 21/474; G16B 50/30; G01N 2021/4742; G01N 2021/4769; G01N 2800/2821; G01N 2800/32

(57) ABSTRACT

A method for identification of amino acid residues in a fluid sample (9) is disclosed. The method comprises producing (100) a light signal from a laser (1) and illuminating (120) the fluid sample (9) with the light signal through a lens in a sensing probe (8). A light signal is acquired (130) from the fluid sample (9) and a plurality of features is extracted (140) from the light signal. The extracted plurality of features is compared with a model in a database to determine and quantify the amino acid residues in the fluid sample (9).

### CROSS REFERENCES TO RELATED APPLICATIONS

This application claims benefit to and priority of LU Patent Application No. LU102007 “DEVICE AND METHOD FOR DETECTING PEPTIDES AND PROTEINS IN A FLUID SAMPLE”, filed on 20 Aug. 2020.

### FIELD OF THE INVENTION

The invention relates to a device for detecting and quantifying molecules with amino acid residues, such as peptides and proteins, in a liquid dispersion sample.

### BACKGROUND OF THE INVENTION

Disease biomarkers are indicators that can be used for diagnostic, prognostic, and even therapeutic purposes. Several molecules with amino acid residues, i.c., proteins and peptides, have been found to be biomarkers found abnormally expressed during the development of diseases. Some of the proteins and peptides have been in use in clinically relevant environments for a long time. For example, in Alzheimer's disease (AD), the most validated pathological biomarkers include the Amyloid-beta (Aβ) peptides, total Tau (T-tau) protein and the hyperphosphorylated form of Tau (P-tau) protein. The detection and quantification of these biomarkers in the biofluids of patients, particularly in cerebrospinal fluid (CSF), in clinical context is a frequent routine procedure to detect AD since the biomarkers allow a non-invasive diagnosis of the disease.

Regarding Parkinson's disease (PD), the most common disease biomarker is related to abnormal aggregates of alpha-synuclein (α-syn) protein, which leads to the development of Lewy bodies and contributes to the disease progression. Other neurodegenerative disorders that can be detected based on the levels of the pathological prion protein (Prp^{Sc}) are prion diseases, for example, the Creutzfeldt-Jacob disease (CJD) [1].

Over the past decades, some clinically relevant biomarkers have also been identified and used in cancer research. One of the most used biomarkers for cancer screening is the prostate-specific antigen (PSA) which is a protein produced in the prostate. The PSA test is often made before more invasive tests are carried out to determine the extent of the cancer in the patient.

Further testing of the patient is indicated only when high levels of PSA are found in the blood. Other well-known tumour biomarkers are cancer-associated antigens, such as CA 125, which is a serum-based marker for ovarian cancer, CA 15-3, a cancer antigen biomarker for breast cancer or CA 19-9, a marker for pancreatic cancer. The carcinoembryonic antigen or CEA is also tested in a variety of cancers, for instance in colorectal cancer. These biomarkers are mainly used to understand the disease progression and evaluate how a patient is responding to the treatment [2].

There are numerous biomarkers already well established within the context of cardiovascular diseases (CVDs), as well as associated with its pathophysiological processes. As an example, blood levels of natriuretic peptides (NP), in particular the B-type natriuretic peptide (BNP) and N-terminal pro-B-type natriuretic peptide (NT-proBNP), are promptly measured in patients with heart failure (HF). These peptides have been contributing to the rapid diagnosis and evaluation of the HF treatment and the disease progression, particularly in emergency scenarios [3]. On the other hand, the gold standard biomarkers used in case of suspicion of acute coronary syndrome (ACS)—resulting for instance from a myocardial infarction (MI)—are regulatory proteins, namely cardiac troponin I (cTnI) and T (cTnT). C-reactive protein (CRP) is an ideal biomarker for inflammation and can be typically associated with a variety of diseases but also works as a good indicator for CVDs development [4].

Clinicians and researchers face a huge challenge when analysing the protein levels of the biomarkers in different samples of biofluids. Currently, the measurements are made using different types of instruments, techniques and protocols which causes a variation in the results obtained by different research institutes, hospitals, and associated laboratories. Thus, there is currently no consensus regarding the physiological concentrations of the tested amino acid residues (peptides or proteins) found in the samples of biofluids found in different patients.

Despite this difference, the criteria for diagnosing a patient suffering, for example, from AD is well established. The criteria are based on the levels of the biomarkers (Amyloid-beta (Aβ) peptides, total Tau (T-tau) protein and the hyperphosphorylated form of Tau (P-tau) protein) found in samples of the CSF of patients. Increased levels of both T-tau and P-tau forms and a decrease of Aβ₁₋₄₂ form and Aβ₁₋₄₂/Aβ₁₋₄₀ ratio is a common phenotype among AD patients. The decreased levels of Aβ₁₋₄₂ in the CSF of AD patients, when compared with healthy controls, can be explained by the increase of senile plaque aggregates in the brain, which reduces the amount of peptide that diffuses to CSF. Indeed, some studies indicate that CSF Aβ₁₋₄₂ levels correlate with amyloid deposition confirmed by PET imaging and that the CSF Aβ₁₋₄₂/Aβ₁₋₄₀ ratio is even a more suitable marker for amyloid PET correlation status [5-6].

Aβ and Tau concentrations are around 10 to 100 times lower in plasma and/or in serum than in the CSF, which means that these biomarkers must be measured in lower ranges in the samples taken from the bloodstream. Nevertheless, T-tau and p-tau protein levels in the samples from the bloodstream are also found to be elevated in AD patients and may therefore also reflect an association with AD pathology. However, further studies are required to provide more evidence that the Tau protein found in the plasma of the bloodstream is an AD specific marker and not a general indicator of neurodegeneration [7-8]. Several studies have also been reporting a decrease in Aβ₁₋₄₂ as well as in the Aβ₁₋₄₂/Aβ₁₋₄₀ ratio in the plasma of AD patients. An association between a lower plasma Aβ₁₋₄₂/Aβ₁₋₄₀ ratio and PET-imaging positivity for amyloid plaques deposition has been also reported, which may justify a future use of this ratio as a predictor of AD. Even though the Tau protein and Aβ₁₋₄₂/Aβ₁₋₄₀ ratio are promising candidates, there is still, as far as our knowledge goes, no blood-based biomarker established for AD diagnosis [9-10].

Current methods to directly identify and quantify the presence of molecules with amino acid residues (such as peptides and proteins) include immunological approaches, such as the enzyme-linked immunosorbent assay (ELISA), and more advanced proteomic methods involving mass spectrometry (MS). A wide range of methods combining these two categories are also available [17].

Immunoassays are widely used and very sensitive approaches. They are based on antigen-antibody interactions requiring the use of quality antibodies to target the protein or the peptide of interest present in the sample. ELISA is a conventional immunoassay procedure and, although it is relatively simple, ELISA can be very time-consuming and also produce false-positive findings due to high levels of non-specific binding. Thus, the ELISA immunoassay procedure lacks specificity. Another disadvantage is the high price of the associated quality antibodies, which makes ELISA expensive. Nevertheless, ELISA has been considered one of the main methods for biomarkers' quantification in biofluids. For instance, a manual method based on standard ELISA (Innotest® ELISA) could detect both Aβ₁₋₄₂ and Aβ₁₋₄₀ in plasma samples with a limit of detection of 7.8 pg/mL [18].

The quantification limits for Aβ₁₋₄₂, Aβ₁₋₄₀ and Tau are, respectively, 5.8 pg/mL, 9pg/mL, and 1 pg/mL for an automated ELISA technique developed by Roche (Elecsys®).

On the other hand, MS based methods are powerful tools involving the identification of several proteins and peptides in a biological sample, based on the analysis of the peaks collected from components' mass spectra (the collected pattern of the mass/charge ratio of ionized molecules). MS techniques provide high specificity, sensitivity, and fast results. However, spectrometers are very expensive instruments. MS-based methods, such as selected reaction monitoring (SRM) have been applied for quantification of Aβ₁₋₄₂, Aβ₁₋₄₀, and Tau biomarkers in CSF samples of AD patients. Analysis by SRM showed a lower limit of quantification (LOQ) for Aβ₁₋₃₈, Aβ₁₋₄₀, and Aβ₁₋₄₂ of 250 pg/mL, 62.5 pg/mL, and 62.5 pg/mL, respectively [20].

Recent studies using other ultrasensitive approaches have been developed for improved quantification biomarkers that are found at very low concentrations in the blood. These methods also use antibodies but achieve results with higher sensitivity and accuracy. Three examples of these methods are: One single-molecule assay (SIMOA technology), the ELISA based sandwich immunoassay (ABtest®) and the immunomagnetic reduction assay (IMR). One study using the SIMOA approach was able to measure plasma concentrations of Aβ₁₋₄₂, Aβ₁₋₄₀, and Tau with a limit of quantification of 0.34 pg/ml, 0.16 pg/ml, and 0.42 pg/mL, respectively [21]. Another study reported a lower LOD using the same SIMOA approach - of 0.019 pg/mL for Aβ₁₋₄₂ and 0.16 pg/mL for Aβ₁₋₄₀ [22]. The ELISA based sandwich immunoassay (ABtest®) achieved a LOD for Aβ₁₋₄₂ in plasma of 3.60 pg/mL and for Aβ₁₋₄₀ a value of 7 pg/mL [23]. A higher sensitivity of detection is reached when using the immunomagnetic reduction assay (IMR). The IMR assays can measure low-detection limits for Aβ₁₋₄₂, Aβ₁₋₄₀, t-Tau and p-Tau of 0.770 pg/mL, 0.170 pg/mL, 0,026 pg/mL, and 0.0196 pg/mL, respectively, using a superconducting quantum interference device (SQUID) [24]. However, these methods are both time-consuming and expensive. In addition, due to the lack of sensitivity of these methods, it is possible that the lowest concentrations may not be detected. In addition, techniques such as ELISAs are not able to distinguish between monomers or oligomers in a single process.

The publication “Optical fibre-based sensing method for nanoparticle detection through supervised back-scattering analysis: a potential contributor for biomedicine” (Paiva et al., in OPTICAL FIBERS AND SENSORS FOR MEDICAL DIAGNOSTICS AND TREATMENT APPLICATIONS XIX, vol. 10872, 27 Feb. 2019 (2019-02-27)) teaches the detection of nanoparticles by back-scattered laser light signal collected by a polymeric lensed optical fibber tip dipped into a solution of synthetic polystyrene nanoparticles. The authors were able to correctly detect the presence of 100 nm synthetic nanoparticles in distilled water at different concentration values. The authors noted in the paper the difficulties that scientists have had in developing a “simple and fast” method to accurately detect and characterise extracellular vesicles. Indeed, the authors of this paper also failed to apply their method to natural, biological materials.

The method and device disclosed in this document enables the detection of molecules made up of amino acid residues, such as peptides and proteins, in sample of biofluids taken from patients at very low concentrations and the discrimination between three peptides even with a similar molecular mass.

### SUMMARY OF THE INVENTION

A method for identification of amino acid residues, such as but not limited to peptides or proteins, in a fluid sample using machine learning techniques is disclosed. The method comprises producing a light signal from a laser, illuminating the fluid sample with the light signal through a lens in a sensing probe, acquiring a light signal from the fluid sample, extracting a plurality of features from the light signal, and comparing the extracted plurality of feature with a model in a database to determine the amino acid residues in the fluid sample.

In one aspect, the method enables the detection of the presence or absence of a specific peptide, the identification of which peptide being detected from other peptides and the quantification of the detected peptide. Both supervised learning methods (e.g., support vector machines, random forests, neural networks, etc) or clustering algorithms/unsupervised methods (e.g., K-means, U-Map) are used for identifying the peptide. Regression models (e.g., random forests regressor, linear regressor, polynomial regressor, etc) can be used for quantifying the peptide. The method can also be used for detection of proteins.

The method further comprises in one aspect the filtering of the acquired light signal to remove noisy low-frequency components and/or normalizing the light signal.

The light signal from the laser is modulated and the extraction of the plurality of features in the light signal is carried out over periods of time. The plurality of features are time domain and frequency derived features.

The model is created by one of a support vector machine or a clustering algorithm. It is also possible to use different models for different purposes.

A device for identification of amino acid residues in a fluid sample is also disclosed in this document. The device comprises a laser which is connected through an optical fibre with a sensing probe (8) with a lens, such as a microlens, for illuminating the sample. A detector acquires a light signal from the sample and a computer is adapted to analyse the light signal, extract features from the light signal, compare the extracted features with stored features in a database and produce a result.

The method and the device can be used for the detection of neurodegenerative disease, such as Alzheimer's disease, cardiovascular diseases, and cancer.

Concentrations above and below the biomarkers' human plasmatic concentrations regarding AD were tested. Two Aβ-derived peptides (with 42 and 28-amino acids) were tested in a concentration range of 1 pM-10 nM (including the Aβ₁₋₄₂ plasmatic concentrations ranging from 5-300 pg/mL, that corresponds to a range between 5-60 pM [11]). T-tau was tested in a concentration range of 0,1 pM-10 nM, considering its reference physiological levels of 4-55 pg/mL (that corresponds to 0.1-10 pM) [7,12]. P-tau was tested in a concentration range of 0.01 pM-10 nM, considering its reference physiological levels of 0.1-1.2 PM [7,12]. The reference physiological levels are those levels at which the biomarkers are expected to be found in physiological samples, such as blood, plasma, and serum. Experiments started from the lowest—in the pM range—to the highest concentrations—until the nM range—to achieve a saturated peptide/protein concentration.

Plasma concentrations of α-synuclein of PD patients also vary between 1.6 to 320 pg/mL, depending on the method of quantification [13-16].

### DESCRIPTION OF THE FIGURES

FIG. 1 shows a schematic of the acquisition apparatus.

FIG. 2 shows a projection of the LP01 mode at the fibre output on a planar surface.

FIG. 3 shows (left) a simple polymeric lens-like tip and (right) a polymeric lens-like tip with a protective structure surrounding the tip.

FIG. 4 shows a block diagram of the modules and interconnections used in this device.

FIG. 5 shows a scheme explaining the peptide detection calibration pipeline.

FIG. 6 shows a scheme explaining the peptide identification calibration pipeline.

FIG. 7 shows the amplitude plots (left) and FFT plots (right) from an acquisition.

FIG. 8 shows a schematic diagram of the apparatus for probe cleaning studies.

FIG. 9 shows a (top) Spectrum variation along 10 consecutive dips in a serum sample followed by a dip in ethanol (70%) and (bottom) Spectrum variation along 10 consecutive dips in a serum sample followed by a dip in bleach (20%) and distilled water.

FIG. 10 shows a spectrum variation along 10 consecutive dips in a serum sample followed by a dip in bleach (20%): (dash line)—spectra acquired immediately after the dip in serum; (bold line)—spectra acquired after the dip in bleach (20%) after the serum dip.

FIG. 11 shows a signal processing pipeline.

FIG. 12 shows the median probability values for the presence of Amyloid-beta 1-42 in human serum (in a dilution of 1:2).

FIG. 13 shows the algorithm accuracy values for the detection of Amyloid-beta 1-42 in human serum (in a dilution of 1:2).

FIG. 14 shows the median probability values for the presence of Amyloid-beta 1-42 in total human serum.

FIG. 15 shows the Amyloid-beta 1-42 detection accuracy values for several concentrations in total human serum.

FIG. 16 shows the Median probability values for the presence of Amyloid-beta 1-28 in human serum (in a dilution of 1:2).

FIG. 17 shows the Amyloid-beta 1-28 detection accuracy values for several concentrations in diluted human serum (1:2).

FIG. 18 shows the Median probability values for the presence of Amyloid-beta 1-28 in total human serum.

FIG. 19 shows the Amyloid-beta 1-28 detection accuracy values for several concentrations in total human serum.

FIG. 20 shows the median probability of peptide presence for Tau 441 in a human serum (dilution 1:2).

FIG. 21 shows for Tau 441 detection accuracy values versus concentration for diluted human serum (1:2).

FIG. 22 shows the median probability values for the presence of Tau 441 in total human serum.

FIG. 23 shows the Tau 441 detection accuracy values versus concentration for total human serum.

FIG. 24 shows the median probability of peptide presence for Phosphorylated Tau 441 in a human serum (dilution 1:2).

FIG. 25 shows for Phosphorylated Tau 441 detection accuracy values versus concentration for diluted human serum (1:2).

FIG. 26 shows a graphical representation of the regression analysis results (Tau 441, total human serum).

FIG. 27 shows a schematic representation of the peptides' dilutions workflow.

FIG. 28 shows an outline of the method of this document.

FIG. 29 shows the median probability of IL-6 presence among different concentrations.

FIG. 30 shows the accuracy for the detection of IL-6 in the different concentration solutions.

FIG. 31 show the median probability of detecting galectin at different concentrations.

FIG. 32 shows the accuracy of detection of galectin in solutions with different concentrations.

FIG. 33 shows predictions made by Amyloid-beta quantification model.

FIG. 34 shows predictions made by IL-6 quantification model and respective error bars.

FIG. 35 shows predictions made by galectin quantification model and respective error bars.

### DETAILED DESCRIPTION OF THE INVENTION

A detailed schematic of the acquisition apparatus is depicted in FIG. 1. The acquisition apparatus comprises an irradiation laser 1 (Lumentum Operations LLC, San Jose, CA, Model #S28-7602-500) emitting at 976 nm wavelength. The laser light from the irradiation laser 1 was modulated to produce a modulation signal in frequency by a sinusoidal signal (fundamental frequency of 1 kHz, to escape from the electrical grid 50 Hz harmonics) digitally generated at a sampling rate of 10 kHz using a custom-build MATLAB script according to the equation:

1.45+0.045*sin(2*π*1000*t), t−time in seconds

Considering the laser driver's gain, the laser characteristic curve, and the optical loss along the fibre components, the lens' output optical power was 40 mW (but this is not limiting of the invention). This value was determined in accordance with the values used in the literature for optical delivery, collection, and manipulation effects through optical fibres considering the selected wavelength value range, and to cause as little damage as possible to the biological human-derived samples [28].

The modulation signal was externally injected into a laser driver 2 (MWTechnologies Lda, Portugal, Model #cLDD) through one of the output digital-to-analog ports of a data acquisition board 3 (NI, Austin, TX, Model #USB-6212 BNC). The resulting optical signal, mirroring the modulation equation, is inserted into an optical fibre and passes through a 1/99 optical coupler 4 (Laser Components GmbH, Germany, Model #3044214). While most of the radiation follows to the rest of the optical circuit, 1% of the radiation is monitored using a silicon photodetector 5 (Thorlabs Inc, Newton, NJ, Model #PDA-32A2) connected to one DAQ analog-input port.

A 50/50, 1×2, optical coupler 6 (AFW Technologies Pty Ltd, Australia, Model # FOSC-1-98-50-L-1-H64F-2) establishes a bidirectional connection between the incoming light from the laser module, a sensing photodetector 7 (Thorlabs Inc, Newton, NJ, Model #PDA-32A2) and a sensing probe 8. The sensing probe 8 is a microlensed optical fibre with its end just outside a metal capillary and is described below. The metal capillary gives stability to the optical fibre and protects the optical fibre to make sure that the optical fibre does not break. This arrangement allows the sensing probe 8 to simultaneously focus the light coming from the laser 1 and a collection of back-scattered radiation arising from a liquid dispersion sample 9 to be analysed. To provide further information about the samples' conditions/properties, temperature readings are obtained using an Infrared Thermometer 10 (Axiomet, Poland, Model #AX-7600).

The arrangement set out above is merely exemplary and is not limiting of the invention. Other optical components could be used.

The sensing probe 8 is manipulated using a 4 axis (x, y, z, and tilt) right-hand micromanipulator 11 (Siskiyou Corporation, Grants Pass, OR, Model #:MX7600) with a probe holder in which the capillary with the sensing probe 8 is fixed. This micromanipulator is connected to a closed-loop dial controller (Siskiyou Corporation, Grants Pass, OR, Model #:MC1000e-R1/4T) that allows a more precise displacement of the sensing probe 8 into and inside the sample 9.

A visualization and imaging module is composed of a self-made inverted microscope setup using a standard white LED light source 12, an objective 13 (currently at 20×, but higher amplification can be used to observe smaller particles), a mirror 14 and a zoom lens 15 (Edmund Optics, Barrington, NJ, Model #VZM 450). This microscope drives the desired imaging plane to a digital camera 16 (Edmund Optics, Barrington, NJ, USA Model EO-1312C #Model 83-770). The image from the digital camera is observed in real-time in a computer 17 using IDS:'s software uEye Cockpit. The sensing region of the digital camera 16 allows for the visualization of the focused infrared beam from the fluid sample 9 and the reaction of the focused infrared beam with the constituents of the fluid sample 9.

The fabrication of the polymeric microlens used in the sensing probe 8 will now be described. The polymeric microstructures used are fabricated through a guided wave photopolymerization process on top of cleaved optical fibres [25-27], a process in which the cross-linking of monomers is triggered by light at a specific wavelength. Two components must be present in the solution for the photopolymerization process taking place, a monomer, and a photo-initiator:
Monomer: pentaerythritol triaclylate (PETIA) (n=1.48).
Photo-initiator: Bis(2,4,6-trimethylbenzoyl)-phenylphosphineoxide (IRGACURE 819)—sensitive to wavelengths between 375 nm and 450 nm.

Once the correct proportion between monomer and photo-initiator is achieved, an optical setup consisting of a couple of mirrors and a CW laser is used to excite the photo-initiator. In this example, a laser was used emitting laser light at a wavelength of 405 nm (Omicron, Rodgay-Dudenhofen, Germany, #Model LuxX cw, 60 mW) is incident at 45° in two consecutive mirrors, resulting in a square shape optical path. After the second reflection, the laser is coupled into an optical fibre by an objective.

The optical fibre (Thorlabs, Newton, New Jersey, USA #Model SM 980-5.8-125) has a multi-mode behaviour for this wavelength, a multitude of optical modes can be excited, resulting in a different optical output pattern and a consequent difference in the geometry imprinted in the tip.

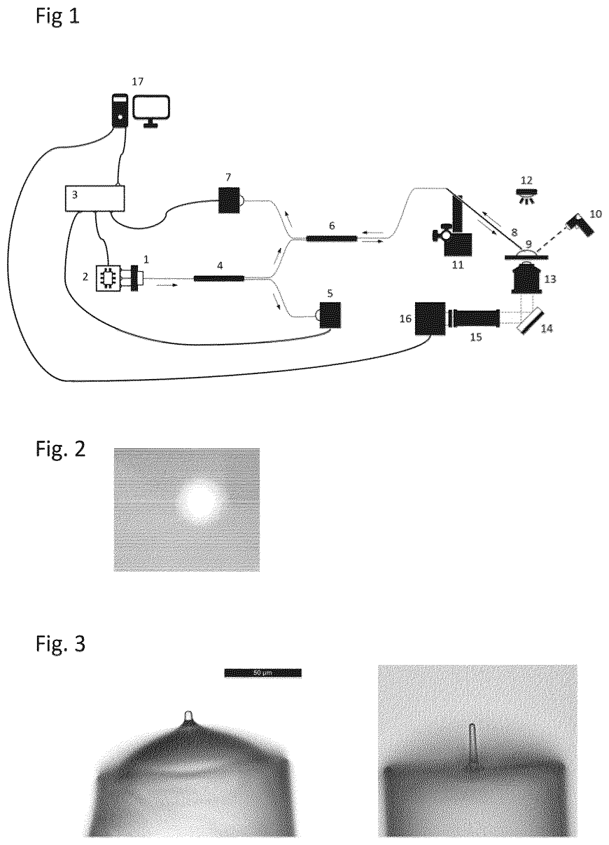

The shape of the structure of the polymeric optical tip should be a substantially spherical, lens-like termination so that the structure of the polymeric optical tip efficiently focuses the incident light. This requires the excitation of a mode with a Gaussian or Gaussian-like profile. Such profiles can be attained with the LP01 and LP02 optical fibre modes as are shown in FIG. 2. Careful alignment of the setup is required to enable the excitement of one of these two optical fibre modes and hence maximum reproducibility.

Once the setup is aligned, i.e., one of the LP01 or LP02 modes is observable at the output of a cleaved fibre (as seen in FIG. 2), the laser is turned off and the fibre is vertically dipped in a drop of the monomer containing a percentage of photo-initiator between 0.2% to 0.5% in weight. When the fibre is retrieved, a drop of solution stays on the apex of the cleaved fibre, and once the laser is turned on the photopolymerization process occurs. Characterized by a self-assembly effect, the process results in a refractive index increase in the areas where the beam is incident, creating a self-guiding effect that will prevent radiation from scattering to other areas of the drop. A 10-seconds exposure is enough to obtain the desired shape. A long exposure period would result in a flat tip surface and not on the desired mode imprint. After rinsing the non-polymerized left off polymer with ethanol (70-96%), the final structure has the diameter of the excited fibre mode and the visual aspect of a spherical lensed tip as depicted in FIG. 3 (left). Given its high aspect ratio (AR), the polymeric optical tip is a very fragile structure by itself. As such, to increase the contact surface and decrease the AR, a protective structure is built around the original polymeric optical tip, assuring a more robust structure. This second step of the fabrication process comprises dipping the already built polymeric optical tip in a new monomer solution containing around 2% of photo-initiator in weight (the same concentration of photo-initiator used for the polymeric optical tip fabrication can also be used in this step). Then a visual verification is conducted to see if the polymeric optical tip's extremity is left outside of the drop. In the cases in which this is verified, the laser is turned on at approximately 20 μW for 5 minutes. When that does not occur naturally, a few drops of ethanol (70-96%) are approximated to the polymeric optical tip, resulting in a rise of the solution drop along the optical fibre, exposing the polymeric optical tip's extremity. Once this is achieved, the exposure proceeds with the same parameters previously mentioned, resulting in a structure like the one presented in FIG. 3 (right).

During the fabrication procedure, some geometrical parameters, such as diameter and length, as well as the curvature radius of the polymeric optical tip are controlled. This can be done through the manipulation of some fabrication parameters, such as the optical fibre mode excited during polymerization, as previously mentioned, but also the percentage of the photo-initiator present in the solution, the exposure time, and laser power used during the polymerization, etc. To assure a high reproducibility of these polymeric optical tips, these parameters should be left constant throughout the whole fabrication process of a batch of polymeric optical tips. The requirements that must be kept constant as well as the parameters to control are summarized in Table 1.

TABLE 1
Requirements and parameters to control during tip production.

| REQUIREMENT | PARAMETERS TO CONTROL |
|---|---|
| SPHERICAL TOP | Excited optical mode |
| SIMILAR TIP RADIUS | Laser Power |
| FOR ALL TIPS | Exposure Time |
| | Photo-initiator concentration |
| SIMILAR TIP LENGTH | Monomer drop left on the fibre |
| FOR ALL TIPS | Laser Power |
| SIMILAR REFRACTIVE | Laser Power |
| INDEX FOR ALL TIPS | |
| SIMILAR PROTECTIVE | Second monomer drop |
| STRUCTURE | Laser Power |
| (GEOMETRY AND | Exposure Time |
| REFRACTIVE INDEX) | Photo-initiator concentration |

For the purposes of the work presented in this text, the fabrication parameters used in the photopolymerization process were the following:
Laser Power (Tip): ≈4 μW
Laser Power (Protection): ≈25 μW
Exposure Time (Tip): 10 s
Exposure Time (Protection): 3 min
Photo-initiator concentration (Tip & Protection): 0.3%

These parameters resulted in structures of the polymeric optical tip with lengths ranging from 30 μm to 50 μm, with the base of the polymeric optical tips having diameters that range from 4 μm to 7 μm, depending on the mode at the fibre's output. Pending on that, the curvature radius of the lens structures also varied between the values of 1.5 μm to 3 μm. The numerical apertures (NA) values range between 0.25 and 0.5 (values evaluated in a water medium) and a focused spot with dimensions of about ⅓^{rd} to ¼^{th} of the base diameter of the lens was obtained. The protective structure does not significantly affect the light propagation in the simple tip underneath the protective structure. The protective increases the contact area between fibre and polymer to the totality of the optical fibre cross-section, improving the mechanical resistance of the polymeric optical tip to the successive media crossings to which the polymeric optical tip will be exposed (e.g. air to plasma, air to serum, etc.). This structure has the aspect of a cupula placed around the initial polymeric optical tip, always having a height lower than the polymeric optical tip itself.

It will be appreciated that the above description is only one method in which the sensing probes 8 used in this disclosure can be fabricated. The method for detecting the molecules with the amino acid residues is not limited to the sensing probes 8 with the polymeric optical tops fabricated using the above fabrication method. Other structures capable of focusing light to a small spot and thus generate an electric field gradient can be used for the method here described. Such structures can be built on the apex or on the side of an optical fibre or on a planar substrate. It will be appreciated that these structures include optical fibre tapers, phase Fresnel plates (fibre or planar), a single nanometric hole, or an array of nanometric holes on a metallic surface, for plasmonic effects. The latter can either be deposited on an optical fibre or on a transparent planar substrate. To summarize, any type of metalens, be it metallic or dielectric, built on an optical fibre or on a planar substrate is suitable for this application. Back-scattered signal and liquid sample temperature acquisition setup. The setup used for

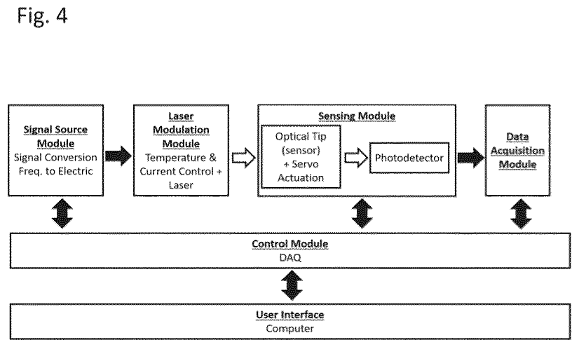

acquiring the back-scattered signal from the liquid dispersion samples 9 using the polymeric optical tip as the sensing probe 9 was comprised of the following modules shown in FIG. 4: a sensing module comprising the lensed optical fibre (the sensing probe 9) inserted into a metallic capillary and manipulated using the 4-axis micromanipulator, two silicon photodetectors 7, and the infrared thermometer 10; a laser module, comprising the laser 1 (976 nm diode laser) and corresponding submodules for laser temperature and current control; a data acquisition module, comprising the data acquisition board (DAQ) 3; a visualization and imaging system, comprising the optical components needed to visualize the optical fibre tip at the micro-scale (i.e. objective 13, mirror 14 and zoom lines 15); and a control unit, for software, hardware controlling and recording and processing the acquired data (the back-scattered signal, the signal collected at the output of the laser and the obtained images). One of the two silicon photodetectors 7 is used to acquire the signal collected at the output of the laser and the other of the two silicon photodetectors 7 is used to acquire the back-scattered signals.

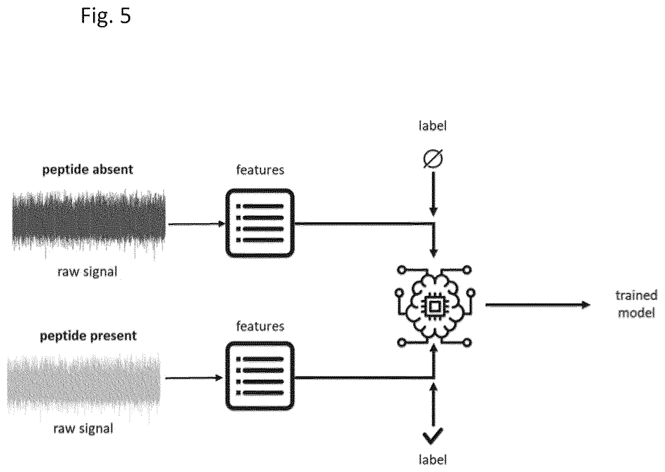

Signal acquisition and processing. After the optical setup for the acquisition apparatus was correctly mounted and turned on, a simple assay was carried out for water/peptides solutions prepared. This is done by placing a volume of 150 μL of the water/peptide solution as the fluid sample 9 over a 35 mm Ibidi® micro rounded dish. Then, the polymeric optical tip of the sensor lens 8 was immersed in this fluid sample 9 with the help of the visualization and imaging system. Different peptides samples acquisition sequences were considered depending on the conducted experiment. The procedure used for calibrating the system regarding the peptide detection functionality is based on the following steps and is shown in some detail in FIG. 5. In a first step, the backscattered signal is acquired from samples with no peptide (as shown in the topmost raw signal of FIG. 5). Then the backscattered signal is acquired with peptides (tau-441, beta-42, beta-28, and phospho-tau) present at different concentrations (shown in an example in the bottommost raw signal of FIG. 5—there will be a number of these). The number of samples for each class should be approximately the same. If the number of samples is not the same, then there is a risk that the results from the classification model will be biased.

In a next, a set of descriptive features are extracted from the signal. The descriptive features are given below. The resulting dataset is used to train a binary classification model. Once the model converges and its generalization capability is assured, the system is ready to make predictions. There are several classification models that can be used for training, and these are explained in more detail below.

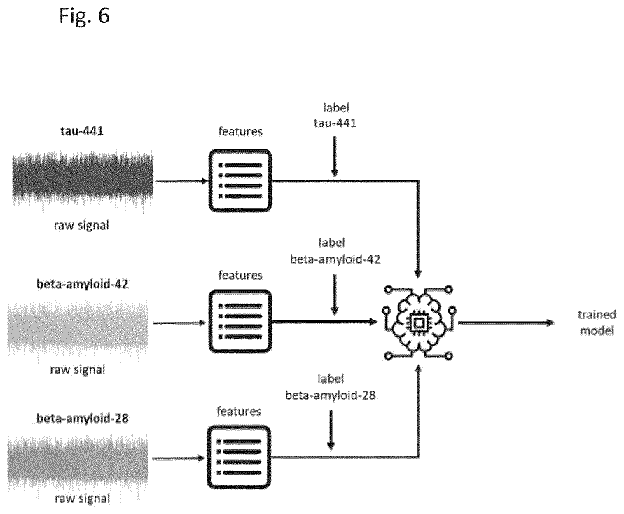

The calibration pipeline applied for creating multiclass artificial intelligence models able to identify the type of the peptides present in the solution is also schematized in FIG. 6. The back-scattered signal provided from the samples spiked with each one of the three different peptides (tau-441, beta-amyloid-42, beta-amyloid-28) were acquired and are shown in FIG. 6. The corresponding descriptive features are calculated, and the features are fed to the classification model together with the samples' class labels (i.e., the identification of the peptides tau-441, beta-42, beta-28, or phospho-tau). Once the model is trained, it is ready to make the predictions.

Temperature sensing based on back-scattered frequency features.

Sample temperature acquisition. The influence of sample's temperature on the back-scattered signal was evaluated through a simple experiment where distilled water was used in replacement of a biofluid (e.g., serum sample) as the fluid sample 9.

A distilled water sample (used as the fluid sample 9) of 1 mL at room temperature was placed in an Ibidi® dish and the back-scattered signal was collected for 30 seconds, 10 times in a row, in different locations of the sample. Time and frequency features were then calculated based on the collected back-scattered signal using the algorithm of this disclosure. The water temperature was measured at the beginning of the acquisitions and once again at the end, to monitor variations, using the infrared thermometer 10. It will be appreciated that this temperature recording could also be done using other automatic means, in particular using a type “T” thermocouple with an automatic logger for the detection of the temperature variation over time within a single sample.

After temperature analysis, the sample 9 was discarded and a new one was pipetted for analysis. This was repeated 10 times for 10 samples of 1 mL of distilled water. All of these samples 9 were collected from the same tube.

### Peptides Detection Among Different Concentration Values/Peptides Quantification

Output laser and back-scattered signals were acquired at first in non-spiked human serum samples (“blank” samples) and then in the human serum samples spiked with the peptide/protein in the pre-selected concentrations used as the fluid sample 9. The data was collected from the fluid samples 9 with the lowest to the highest peptide/protein concentration and two human serum dilutions. This sequence was considered for Amyloid-beta 1-42, Amyloid-beta 1-28, Tau-441 and Phosphorylated Tau 441. A cleaning protocol of the sensor probe 8 was applied for data collection between different human serum dilutions.

The peptides' concentration test was conducted for four different peptides, namely the Aβ1-42, the AB1-28, the Tau-441 and the Phosphorylated Tau 441. The tested concentrations for each of the peptides were determined considering the typical physiological concentration in humans. Given that these are different for the Amyloid-beta and the Tau peptides, a different selection of test concentrations present in the human serum sample was made. These are depicted in Table 1.

Table 1—Peptides' concentrations analysed during the detection experiments, presented in picomolar (pM). The order of the analysis followed the increase in concentration values.

| | Peptide concentration (pM) | | | | | | | | | | | | |
|---|---|---|---|---|---|---|---|---|---|---|---|---|---|
| Aβ₁₋₄₂ | 0 | 0.01 | 0.1 | 1 | 5 | 25 | 50 | 100 | 1000 | 10000 | 100000 | 1000000 | 10000000 |
| Aβ₁₋₂₈ | 0 | — | — | 1 | 5 | 25 | 50 | 100 | 1000 | 10000 | — | — | — |
| Tau-441 | 0 | — | 0.1 | 1 | 10 | — | — | 100 | 1000 | 10000 | — | — | — |
| Phosphorylated | 0 | 0.01 | 0.1 | 1 | 10 | — | — | 100 | 1000 | 10000 | — | — | — |
| Tau 441 | | | | | | | | | | | | | |

All the concentrations in Table 1 were tested twice, from the lowest concentration (0 pM) to the highest (10000 pM), using a single probe for each of the peptides. The first sequence had the serum samples diluted in PBS at a 1:2 ratio, and the second made use of non-diluted serum, here defined as a 1:1 ratio. Between these two dilutions, a cleaning protocol was applied (see below) to prevent cross-contamination from one sample to the other.

Additionally, higher concentrations of Amyloid-beta 1-42 peptide were also tested, namely the 1 nM, 5 nM, 25 nM, 50 nM, 100 nM, 1000 nM, and 10000 nM. As described above, both dilutions were tested (1:2 and 1:1), from the lowest to the highest concentration.

### Classification/Distinction of Different Peptides

To perform the distinction analysis, the same sensing probe 8 was exposed to serum solutions containing the same concentration of different peptides. The used peptides were the same as in the previous tests, the Amyloid-beta 1-42, Amyloid-beta 1-28 and the Tau-441, only this time, the tested concentrations were the same for all the peptides, them being 0 pM, 1 pM, 10 pM, 100 pM, and 1 nM. Each of the three peptides was tested for each concentration value (from the lowest to the highest), beginning with Amyloid-beta 1-42, followed by Amyloid-beta 1-28 and, finally, by Tau-441. Once again, the analysis sequence considered included at first the 1:2 serum dilution in PBS and then the non-diluted serum. As the sensing probe 8 was consecutively exposed to different peptides, the cleaning protocol (see below) was applied after each acquisition, to prevent cross-contamination from affecting the results.

### Back-Scattered and Laser Output Signal Acquisition

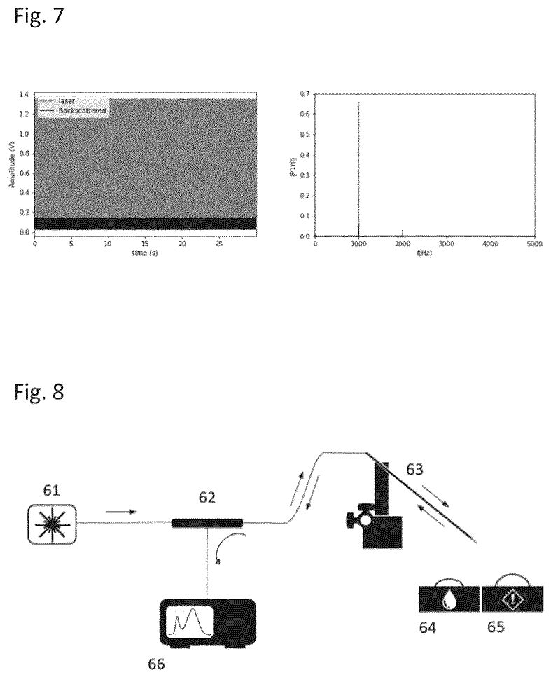

The laser output and backscattered signals were acquired simultaneously by a custom-built MATLAB script (as noted above) which, after a starting order, records and saves the input from both photodetectors for 30 seconds, at 10 kHz sampling rate. The scrip also plots the acquired signals (FIG. 7, left) and their FFTs (Fast Fourier Transforms) (FIG. 7, right), allowing for an immediate visual analysis of the experiment's results.

To avoid sample misrepresentation and ensure statistical variability, for every sample, 10 acquisitions were performed at different locations, following the above-mentioned script.

### Cleaning Protocol

To prevent cross-contamination between samples, a standard cleaning protocol was followed. The sensing probe 8 was inserted into a solvent (e.g., diluted bleach) between any two samples 9 to remove any biological traces. Then, the sensing probe 8 was dipped in distilled water to remove any trace of bleach. While in the distilled water, one to two signal acquisitions (as above) were performed to ascertain any degradation issues and ensure probe prime conditions.

The choice of this cleaning protocol was based on a spectral analysis performed to the polymeric tips in the sensing probe 8 after being exposed to different media. The apparatus used for this study is schematized in FIG. 8: light from a C-band (1530-1565 nm) source (61: NetTest Photonics Division (ex-Photonetics), Denmark, Model #:Fiberwhite-SP), coupled into the optical fibre is directed by an optical circulator 62 into the sensing probe 8, as described above, which is dipped into a drop of human serum (sample 9) and then into a drop of a cleaning solvent 65. After this cleaning procedure, the spectral response of the system is acquired by an Optical Spectrum Analyzer (6: Yokogawa Electric Corporation, Japan, Model #AQ6370C).

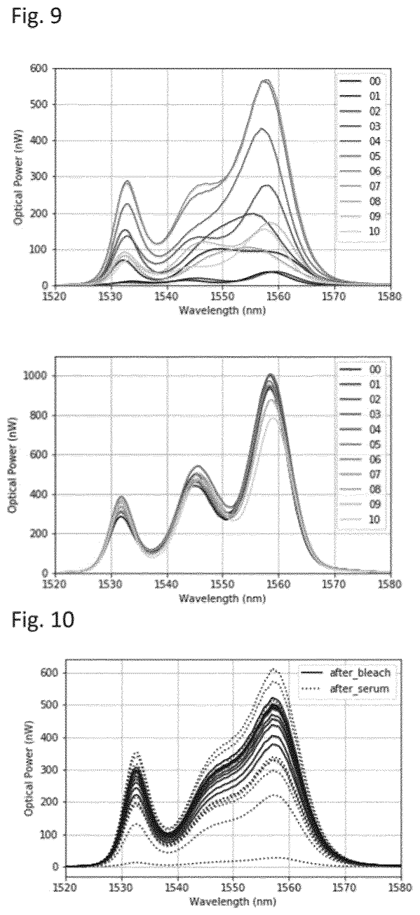

It was observed that when using a solvent such as ethanol (70% diluted in water) after the polymeric optical tip being in contact with the sample 9 of a serum, a deterioration of the polymeric optical tip's reflection spectrum is observed—See FIG. 9 (a). This is a consequence of the fixation of proteins or other biological debris that are present in the fluid sample 9 to the polymeric optical tip, affecting the light propagation and the polymeric optical tip geometrical integrity as well. When changing the protocol to the application of bleach (20% diluted in water), no significant deterioration is verified, and the spectrum's shape remains unaltered throughout the consecutive contacts with the serum (see FIG. 9b). In practice, this means that the bleach is preventing protein/biological debris fixation and thus, cleaning the polymeric optical tip. This becomes clear once the spectra acquired immediately after the serum and the ones acquired after the tip is cleaned with bleach (20%) are overlapped in the same plot—see FIGS. 7 and 8. A bigger variation in values is observed in the spectra collected right after the serum, indicating some deterioration or debris accumulation that affects the radiation. Once the tip is cleaned with bleach, a recovery is observed in the spectrum shape, hence the proximity of all the bold lines (FIG. 10).

Note that the cleaning of the polymeric optical tips can be done either by a chemical or a physical process. Although the present procedure is based on the use of a chemical solvent, the application of a surface treatment capable of preventing proteins adsorption by the surface is also a viable option as well as the application of an ultrasound-based cleaning protocol.

### Back-Scattered and Output Laser Signal Processing

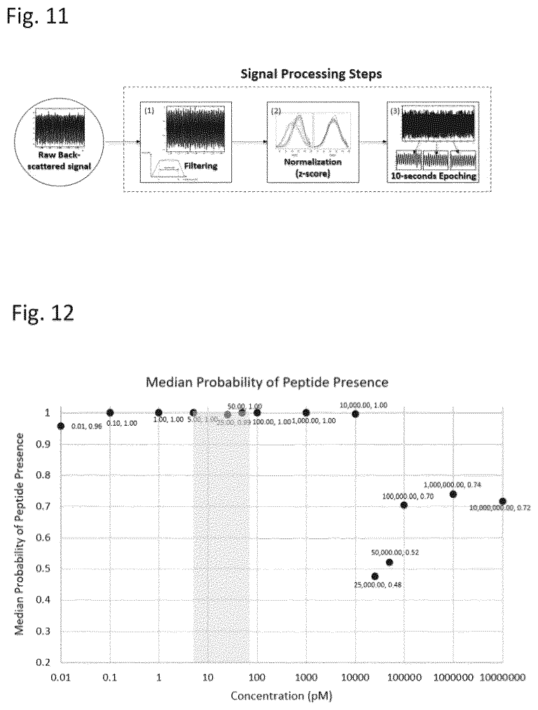

For all the experiments conducted, the back-scattered signals were processed using the same pipeline, schematized in FIG. 11. These steps were applied to each raw signal acquisition set, before extracting the features which characterize the fluid samples 9 and applying any supervised or unsupervised learning method. A custom-built Python 3 script was created for running this pipeline, using the numpy and scipy libraries.

Each acquisition was first filtered using a second-order 500 Hz Butterworth high-pass filter to remove noisy low-frequency components of the acquired signal (e.g., 50 Hz electrical grid component). Then, the signal of each acquisition was normalized using the z-score. The z-score can be calculated using the following equation:

$$z = \frac{x - mean ( x )}{SD ( x )}$$

where mean(x) and SD(x) represent, respectively, the signal average and standard deviation. After this transformation, each whole acquisition was split into epochs of 10 seconds. Features were calculated for each one of these epochs. An additional pre-processing step was tested, which consisted in the subtraction of the laser output to the raw signal.

### Artificial Intelligent (AI)-Based Methods for Peptides Detection and Quantification

Features. After processing the signal of each acquisition, a set of 98 features were calculated for each 10 second epoch (table 3). These features can be divided into two types: time and frequency derived. Within the time domain features it is possible to group them into time domain metrics and non-linear. On the other hand, frequency related features can be subdivided in wavelet packet decomposition, Discrete Cosine Transform (DCT)-derived and spectral features. The feature extraction step was implemented with a custom-built python 3 script, using the scipy, pandas, PyWavelets, librosa, and numba python libraries.

TABLE 3
Calculated features.

| Type | Group | Feature |
|---|---|---|
| Time- | Time | Standard Deviation |
| domain | domain | Interquantile range |
| | metrics | Kurtosis |
| | | Skewness |
| | | Mean |
| | | Root mean square |
| | | Signal power |
| | | Entropy |
| | | Root sum of squares level |
| | | Area under the curve histogram |
| | Non- | Approximate entropy |
| | linear | Singular value decomposition |
| | | entropy |
| | | petrosian fractal dimension |
| | | Higuchi fractal dimension |
| | | Detrended fluctuation |
| | | analysis coefficient |
| | | Hurst Exponent |
| | | Hjorth complexity |
| | | Hjorth mobility |
| Frequency- | DCT- | 1st DCT coefficient |
| domain | derived | 2nd DCT coefficient |
| | | 3rd DCT coefficient |
| | | 4th DCT coefficient |
| | | 5th DCT coefficient |
| | | 6th DCT coefficient |
| | | 7th DCT coefficient |
| | | 8th DCT coefficient |
| | | 9th DCT coefficient |
| | | 10th DCT coefficient |
| | | 11th DCT coefficient |
| | | 12th DCT coefficient |
| | | 13th DCT coefficient |
| | | 14th DCT coefficient |
| | | 15th DCT coefficient |
| | | 16th DCT coefficient |
| | | 17th DCT coefficient |
| | | 18th DCT coefficient |
| | | 19th DCT coefficient |
| | | 20th DCT coefficient |
| | | 21st DCT coefficient |
| | | 22nd DCT coefficient |
| | | 23rd DCT coefficient |
| | | 24th DCT coefficient |
| | | 25th DCT coefficient |
| | | 26th DCT coefficient |
| | | 27th DCT coefficient |
| | | 28th DCT coefficient |
| | | 29th DCT coefficient |
| | | 30th DCT coefficient |
| | | Number of DCT coefficients that |
| | | capture 98% of the original signal |
| | | Total spectrum Area Under Curve |
| | | Spectral Entropy |
| | | 1st Hilbert peak |
| | | 2nd Hilbert peak |
| | | 3rd Hilbert peak |
| | | 4th Hilbert peak |
| | | 5th Hilbert peak |
| | | 6th Hilbert peak |
| | | 7th Hilbert peak |
| | | 8th Hilbert peak |
| | | 9th Hilbert peak |
| | | 10th Hilbert peak |
| | | Number of Hilbert coefficients that |
| | | capture 98% of the original signal |
| | | Haar Relative Power 1st level |
| | | Haar Relative Power 2nd level |
| | | Haar Relative Power 3rd level |
| | | Haar Relative Power 4th level |
| | | Haar Relative Power 5th level |
| | | Haar Relative Power 6th level |
| | | Db10 Relative Power 1st level |
| | | Db10 Relative Power 2nd level |
| | | Db10 Relative Power 3rd level |
| | | Db10 Relative Power 4th level |
| | | Db10 Relative Power 5th level |
| | | Db10 Relative Power 6th level |
| | | Symlet Relative Power 2nd level |
| | | Symlet Relative Power 3rd level |
| | | Symlet Relative Power 4th level |
| | | Symlet Relative Power 5th level |
| | | Symlet Relative Power 6th level |
| | | Db4 Relative Power 2nd level |
| | | Db4 Relative Power 3rd level |
| | | Db4 Relative Power 4th level |
| | | Db4 Relative Power 5th level |
| | | Db4 Relative Power 6th level |
| | Spectral | Spectral contrast std |
| | | Spectral contrast mean |
| | | Spectral contrast max |
| | | Spectral roll-off frequency std |
| | | Spectral roll-off frequency mean |
| | | Spectral roll-off frequency max |
| | | Spectral flatness std |
| | | Spectral flatness mean |
| | | Spectra flatness max |
| | | Spectral centroid std |
| | | Spectral centroid mean |
| | | Spectra centroid max |

### Time-Domain Derived Features.

Time domain metrics such as mean, standard deviation, root mean square, signal power, root sum of squares level (RSSQ), skewness, kurtosis, interquartile range, and entropy were used, given its adequacy in differentiating types of periodic signals. The skewness reflects the distribution symmetry degree while kurtosis quantifies whether the shape of the data distribution matches the Gaussian distribution. The interquartile range is a variability measure. Additionally, the area under the curve of the histogram distribution of the voltage values was considered.

Non-linear features are useful to describe the complexity and regularity of a signal and are often used to describe the phase behaviour of predominantly stochastic signals, such as EEG. A total of eight non-linear features were considered: approximate entropy, singular value decomposition (SVD) entropy, Petrosian fractal dimension, Hurst exponent, Detrended fluctuation analysis (DFA), Higuchi fractal dimension, Hjorth complexity and mobility. The approximate entropy is used to quantify the amount of regularity and the unpredictability of fluctuations over time-series data, whereas the SVD entropy is an indicator of the number of eigenvectors that are necessary for an adequate explanation of the data set, in other words, it measures the dimensionality of the data.

The term fractal relates to fluctuations in time that possess a form of self-similarity whose dimension cannot be described by an integer value. Therefore, a fractal dimension (FD) is a ratio that provides a statistical index of complexity and the degree of irregularity of a waveform. It is a highly sensitive measure for the detection of hidden information contained in physiological time series. Petrosian's algorithm provides a fast computation of the FD of a signal by translating the series into a binary sequence, while Higuchi is iterative in nature and is especially useful to handle waveforms as objects. Finally, DFA is a method for quantifying fractal scaling and correlation properties in the time-series.

The Hurst exponent is a measure of the “long-term memory” of a time series. It can be used to determine whether the time series is more, less, or equally likely to increase if it has increased in previous steps. Hjorth parameters are indicators of the statistical properties of a signal in the time domain. The mobility parameter is defined as the square root of the ratio of the variance of the first derivative of the signal and that of the signal. It represents the mean frequency or the proportion of standard deviation of the power spectrum. On the other hand, the complexity parameter indicates how the shape of a signal is similar to a pure sine wave, this value converges to 1 as the shape of the signal gets more similar to a pure sine wave.

### Frequency-Domain Derived Features

Regarding the frequency-domain analysis of the back-scattered signal, three sets of features were extracted: Discrete Cosine Transform (DCT) parameters, Wavelet derived coefficients and spectral features. The DCT was applied to each epoch. The DCT can capture minimal periodicities of the signal, without injecting high-frequency artifacts in the transformed data. Besides being highly adequate to short signals, it is highly attractive for this type of problems which require to differentiate target classes, because DCT coefficients are uncorrelated. Thus, they can be used as suitable features for characterizing each peptide class. Additionally, the DCT can embed most of the signal energy into a small number of coefficients. The first n coefficients of the DCT of the scattering echo signal are defined by the following equation:

$${E}_{i}^{DCT} [ l ] = {\sum }_{k = 0}^{N - 1} {ε}_{i} [ k ] cos [ \frac{π l ( 2 k + 1 )}{2 N} ] , for l = 1 , … , n$$

where εᵢ is the signal envelope estimated using the Hilbert transform. The following features were extracted from DCT analysis: the number of coefficients needed to represent about 98% of the total energy of the original signal, the first 30 DCT coefficients, the Area Under the Curve (AUC) of the DCT spectrum for all the frequencies before the modulation frequency (1 kHz) and, the entropy of the DCT spectrum. A similar analysis was conducted using the Hilbert transform. The Hilbert transform when applied to the signal produces an analytical real-valued representation of it. The 10 highest amplitude peaks of the Hilbert transformed signal were used as features, as well as the number of coefficients needed to represent about 98% of the total energy of the original signal.

Some parameters based on the information extracted from Wavelet analysis of each original signal portion were also considered as features. Using Wavelet packet decomposition, it is possible to extract, in each frequency band, certain tonal information of the original signal depending on the frequency range and content of the back-scattered signal. For this process, it is necessary to choose a suitable mother Wavelet, that will be used as a prototype to be compared with the original signal and extract frequency subband information. Four mother Wavelets—Haar, Daubechies (Db10 and Db4) and Symlet—were selected to characterize the backscattered signal portions. Six features for each type of mother Wavelet based on the relative power of the Wavelet packet-derived reconstructed signal (one to six levels) were considered.

Spectral features characterize the signal's power spectrum, which is the distribution of power across the frequency components composing that signal. It is obtained using the Fourier Transform. Four measures were derived from the spectrum: spectral flatness, spectral centroid, spectral contrast and spectral roll-off. A total of twelve features were calculated from these measures. The spectral contrast is defined as the difference between valleys and peaks in a spectrum. For each sub-band, the energy contrast is estimated by comparing the mean energy in the top quantile (peak energy) to that of the bottom quantile (valley energy). The spectral flatness (or tonality coefficient) quantifies how much noise-like a signal is. A high spectral flatness (closer to 1.0) indicates that the spectrum is like white noise. The spectral roll-off frequency is defined as the centre frequency for a spectrogram bin such that at least 85% of the energy of the spectrum is contained in this bin and in the bins below. Finally, the spectral centroid indicates where the centre of mass of each frequency bin in the spectrogram is located. For each one of these measures three features were calculated: the mean, the maximum and the standard deviation.

### Temperature Sensing Based on Back-Scattered Frequency Features

The relationship between the temperature and the frequency features was studied by calculating the correlation between the temporal evolution of the features and the temperature variation throughout the experiment. Correlation values were calculated considering the average temperature between the sample's initial and final temperatures along each acquisition. Similarly, the mean value of each feature was calculated for each acquisition, so that the two time-series to be compared (temperature and each light scattered-derived feature) had the same number of points. The correlation was calculated using the following formula:

$${r}_{xy} = \frac{\sum ( {x}_{i} - mean ( x ) ) ( {y}_{i} - mean ( y ) )}{\sqrt{\sum {( {x}_{i} - mean ( x ) )}^{2} {( {y}_{i} - mean ( y ) )}^{2}}}$$

where xᵢ represents the temperature time-series values and yᵢ the feature values. Each time-series was normalized so that the correlation value lies between 0 and 1.

### Peptides Detection Among Different Concentration Values

Two different artificial intelligence pipelines were developed to detect the presence of peptides. The first makes use of a supervised machine learning model—Support Vector Machine, whereas the second uses a clustering technique—U-map.

Supervised Learning Pipeline. The model was trained to distinguish between the presence and absence of the different peptides in the solutions (binary problem). A distinct model was built to detect each one of the peptides. The “absence class” was composed by acquisition samples of serum without the spiked peptide, whereas the “presence class” was composed of acquisitions samples of serum with the added peptide in different concentrations, depending on which peptide ought to be detected. Since the “absence class” had a significantly smaller number of samples, the “presence class” was randomly under-sampled, to build a balanced training set. The samples discarded during the under-sampling process were integrated into the test set. The model used to perform the classification was the Support Vector Machine (SVM) since it is capable to deal either with linear and non-linear input data and it is very suitable for high-dimensionality problems. SVM can distinguish between two different groups by finding a separating hyperplane with the maximal margin between the classes. Three general attributes define the SVM classifier: C—a hyper-parameter which controls the trade-off between margin maximization and error minimization, the kernel—a function that maps the training data into a high-dimensional feature space and, the sigma, which controls the size of the kernel. Several combinations of these parameters were tested to find the optimal model. Each model was trained using a cross-validation strategy. The optimal model was chosen based on the accuracy across all the validation folders.

### Performance Evaluation

Since each acquisition was divided into epochs and the features calculated from these epochs were fed into the AI model, a prediction was made for each one of the epochs. However, the goal was to evaluate the performance of the model in detecting the presence of the peptide at different concentrations. Thus, three different methods can be considered to calculate this performance.

First method: accuracy of the binary classification considering each epoch for each concentration.

Second method: median probability of detecting the peptide across all the samples corresponding to the same concentration.

Third method: obtained through the plot of the histogram of the predicted detection probabilities across all samples. The performance for each concentration is the bin with the most counts, that corresponds to the most frequently predicted probability range. Unsupervised Learning/Clustering pipeline

An unsupervised machine learning pipeline was developed to investigate whether it is possible to detect the presence of peptides without any previous knowledge about the data/any previous training stage. The algorithm comprises a dimensionality reduction using UMAP followed by an HDBSCAN clustering. UMAP is an algorithm for dimension reduction based on manifold learning techniques and concepts from topological data analysis. The first phase of UMAP consists of building a fuzzy topological representation. The second phase is simply optimizing the low dimensional representation to have as close a fuzzy topological representation as possible as measured by cross-entropy. The output of the UMAP is a two-dimensional representation of the feature map. HDBSCAN clustering is then applied to this reduced feature space. HBDSCAN is a hierarchical clustering algorithm that extracts a flat clustering based on the stability of the clusters. At the end, two clusters representing the presence and absence of peptide are provided as an output of the model.

### Classification/Distinction of Different Peptides

The peptide distinction/classification algorithm was based on a supervised learning approach. A random forest classifier was trained to identify the three different peptides (Amyloid beta 1-42; Amyloid beta 1-28 and Tau 441). Random forest consists of many individual decision trees that operate as an ensemble. A decision tree is a flow-chart-like structure, where each internal node denotes a test on a feature, each branch represents the outcome of a test, and each leaf node holds a class label. A tree is built by splitting the source set, constituting the root node of the tree into subsets. The splitting is based on a set of splitting rules based on classification features. This process is repeated on each derived subset in a recursive manner. The recursion is completed when the subset at a node has all the same values of the target variable, or when splitting no longer adds value to the predictions. Each individual tree in the random forest spits out a class prediction and the class with the most votes becoming the model's prediction. Five general parameters that define the random forest were optimized: the maximum depth of the forest, the parameters controlling the number of samples in the leaf and split nodes, the number of features to consider when looking for the best split, and the number of decision trees in the forest. Several models with different combinations of these parameters were trained using a cross-validation strategy. The optimal set of parameters were the ones that produced the model with the higher accuracy across all validation folders.

The dataset was composed by samples of three different peptides (Amyloid beta 1-42; Amyloid beta 1-28 and Tau 441) at four different concentrations (1 pM, 10 pM, 100 pM and 1000 pM). The samples were divided randomly into training and test sets with a 7:3 proportion.

### Performance Evaluation

The performance in the test set was evaluated using the accuracy score and the f1-score. The accuracy score measures the proportion of correct predictions made by the model. The F1-score is a weighted average of the precision and recall. The precision gives the proportion of positive predictions that are actually true, whereas recall measures the proportion of positive samples that are actually predicted as positive. The f1-score is commonly used to evaluate the performance in multiclass problems.

### Peptide Quantification

The concentration of the peptide was determined using a supervised learning model: the random forest. A random forest regressor works similarly to a classification one: it constructs a multitude of decision trees and outputs the mean prediction of the individual trees. For this reason, the same parameters were optimized to choose the best model. A cross validation strategy was used to train the model. The model performance was evaluated using the r² coefficient.

The dataset was constituted by samples of Tau 441 in different concentrations (0 pM, 1 pM, 10 pM, 100 pM)—that matched the human plasmatic levels and above. The concentration values were converted to the logarithmic range, so that the increase in concentration assumed a linear trend. The training and test samples were divided randomly. The training set encompassed 70% of the samples, while the test set represented 30%.

### Performance Evaluation

The error in the regressor prediction was measured using the root mean squared error of the logarithmic concentration values.

$$RMSE = \sqrt{{\sum }_{i}^{N} {( {Predicted}_{i} - {Actual}_{i} )}^{2}}$$

### Results

Temperature sensing based on back-scattered frequency features

Table 4 depicts the most correlated features (r>70%) with the temperature evolution. The correlation with the features derived from the difference signal (output laser subtracted to the back-scattered signal) was significantly smaller, which may be attributed to the fact that the laser and the acquired signal are not completely synchronous. The most correlated feature is the maximum spectral flatness, which suggests that the variation in temperature may influence the spectral content of the signal.

Table 4—Correlation values of the most correlated features with the temperature variation (r>70%). These features were calculated using the back-scattered signal and the signal resulting from the difference between the back-scattered signal and the laser signal output.

| | Correlation with | Correlation with |
|---|---|---|
| | temperature (back- | temperature |
| | scattered signal) | (difference signal) |
| Feature | r | r |
| skewness | 0.722 | 0.202 |
| centroid mean | 0.789 | 0.019 |
| centroid std | 0.853 | 0.342 |
| centroid max | 0.779 | 0.254 |
| spectral roll-off frequency mean | 0.802 | 0.394 |
| spectral roll-off frequency max | 0.798 | 0.307 |
| spectral flatness mean | 0.753 | 0.406 |
| spectral flatness max | 0.898 | 0.513 |
| spectral flatness std | 0.951 | 0.420 |
| spectral contrast std | 0.766 | 0.392 |
| spectral contrast mean | 0.779 | 0.159 |

### Peptides Detection Among Different Concentration Values

The results of the peptide detection differ depending on the algorithm applied. Thus, the results discussion was divided into two sections: the results regarding the supervised learning approach and the ones obtained using the clustering pipeline.

### Supervised Learning

Amyloid-beta 1-42 (Serum dilution 1:2)

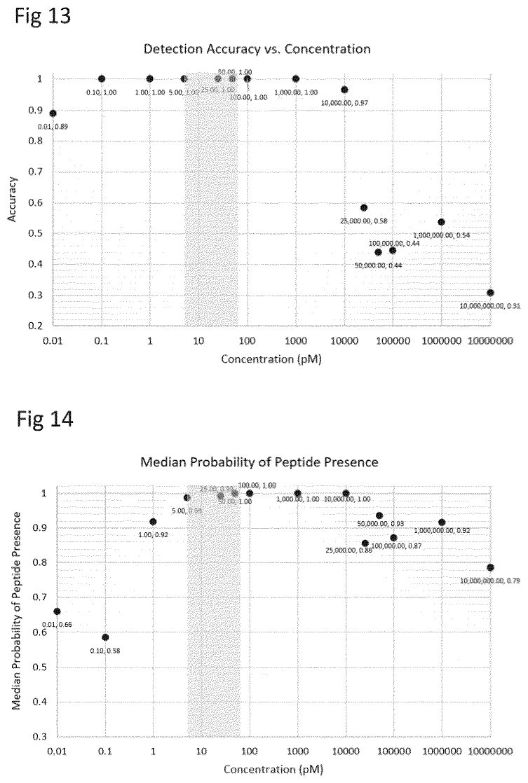

FIGS. 12 and 13 represent, respectively, the ‘Median probability of Peptide Presence’ and ‘Detection Accuracy’ with peptide concentration (in pM) for Amyloid-beta 1-42 in diluted serum (1:2 ratio). The known physiological Amyloid-beta 1-42 concentration range falls within the shaded area (between 5 and 60 pM). The median probability of peptide presence is higher than 99% within the physiological range. As for the detection accuracy, it is near 100% from 0.1 pM to 10000 pM, decaying slightly in the smallest concentration (89% at 0.01 pM), and more abruptly for higher concentrations. This last result is likely attributed to the saturation of the detecting capabilities due to multiple scattering effects.

### Amyloid-Beta 1-42 (Total Serum)

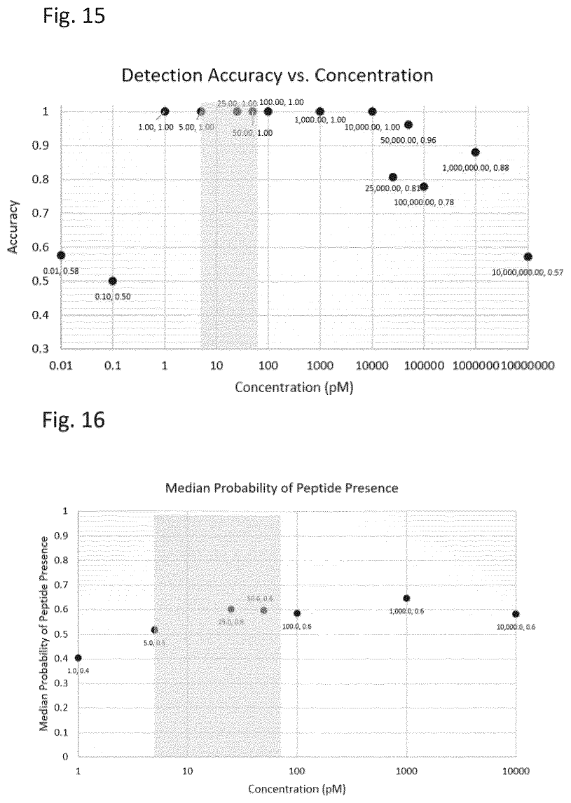

FIGS. 14 and 15 depict, respectively, the ‘Median Probability of Peptide Presence’ and ‘Detection Accuracy’ with peptide concentration (in pM) for Amyloid-beta 1-42 in total serum. The known physiological Amyloid-beta 1-42 concentration range falls within the shaded area (5-60 pM). These results follow the same evolution as for the diluted serum, discussed above, exhibiting 92-100% performance in the 1 pM-10000 pM range, and a slight decay for lower and higher concentrations. In this case, however, the median probability and detection accuracy are smaller for small concentrations. This was expected since the non-diluted serum has more complex molecules in higher concentrations, thus making it harder to detect smaller concentrations of peptide.

Amyloid-Beta 1-28 (Serum dilution 1:2)

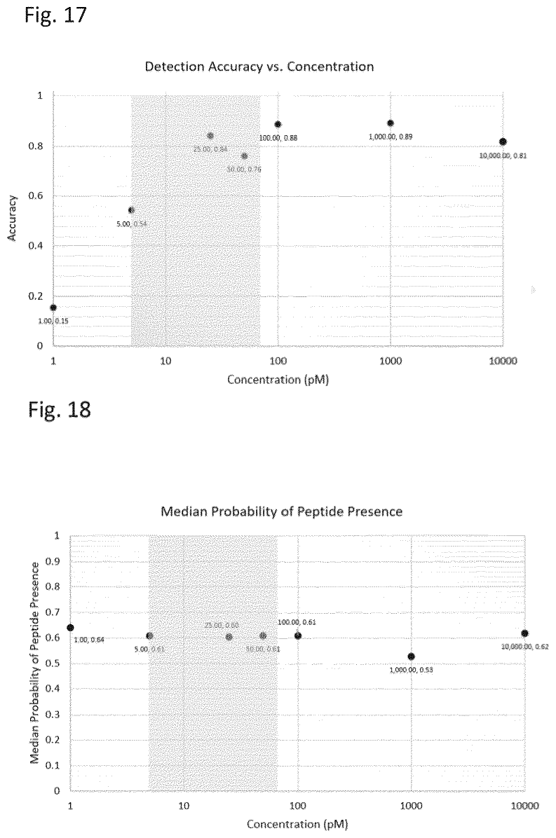

FIGS. 16 and 17 depict, respectively, the ‘Median Probability of Peptide Presence’ and ‘Detection Accuracy’ with peptide concentration (in pM) for Amyloid-beta 1-28 in diluted serum (1:2 ratio). The known physiological concentration range is represented by the shaded area (5-60 pM). The median probability of peptide presence is around 60% in the considered range, with a slight decay for smaller concentrations. The detection accuracy follows a similar pattern, increasing with the concentration and stabilizing in the 80-90% range for concentrations higher than 25 pM. The difference between the median probability and accuracy values can be explained by the method used to calculate the performance. The accuracy considers the performance of the binary classification, independently of the prediction probability. This means that although the model is not very certain about the correct prediction label, it is capable of distinguish the presence of the peptide in most of the epochs.

### Amyloid-Beta 1-28 (Total SERUM)

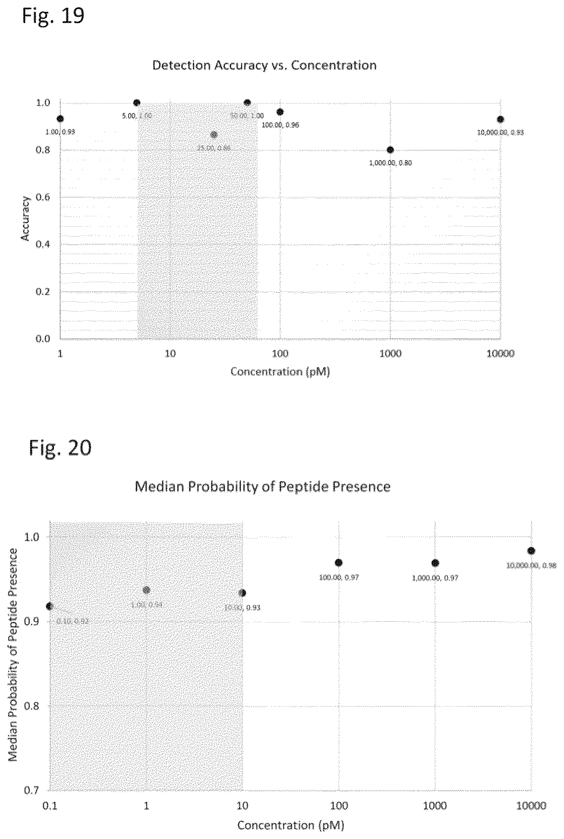

FIGS. 18 and 19 represent, respectively, the ‘Median Probability of Peptide Presence’ and ‘Detection Accuracy’ with peptide concentration (in pM) for Amyloid-beta 1-28 in a non-diluted serum (1:1 ratio). The known physiological Amyloid-beta 1-28 concentration range falls within the shaded area. The overall value of the median probability of peptide presence remained constant around 60% for the evaluated concentrations, with an exception for the 1000 pM, where it drops for 53%. As for the detection accuracy, all the values are above 80%. The same discrepancy between the values of the median probability and the accuracy was observed. This is explained by the same reasoning as for the diluted peptide. These results reflect the capability of the method to successfully identify the peptide's presence in the sample. Nonetheless, these probabilities are lower than the ones observed for the Amyloid-beta 1-42, which can be justified by the smaller dimensions Amyloid-beta 1-28 peptide (molecular size).

### Tau 441 (Serum Dilution 1:2)

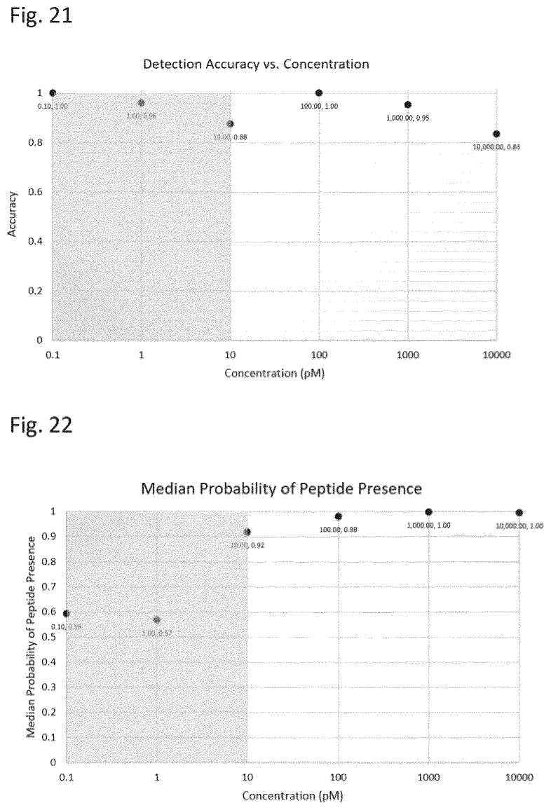

FIGS. 20 and 21 represent the ‘Median Probability of Peptide Presence’ and ‘Detection Accuracy’ with peptide concentration (in pM) for Tau-441 in serum diluted in PBS (1:2 ratio), respectively. The known physiological Tau-441 concentration range falls within the shaded area (0.1-10 pM). The median probability of peptide presence is higher than 90% in the considered range. Detection accuracy is above 80% for all concentration values. It presents a slight oscillation between two maxima for the 0.1 pM and the 100 pM and it slightly decays after this concentration value.

### Tau 441 (Total Serum)

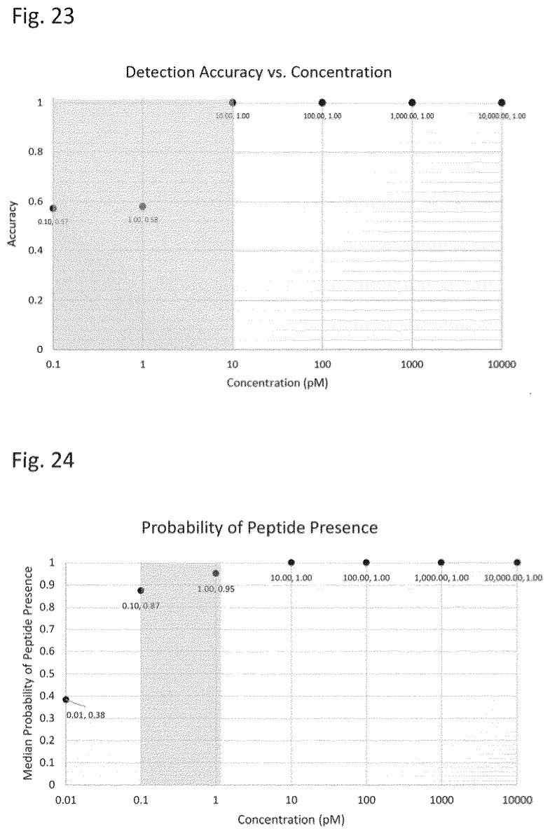

FIGS. 22 and 23 represent, respectively, the ‘Median Probability of Peptide Presence’ and ‘Detection Accuracy’ with peptide concentration (in pM) for Tau-441 in non-diluted serum (1:1 ratio). The known physiological Tau-441 concentration range falls within the shaded area. The median probability of peptide presence increases with the concentration. For concentrations between 0.1 pM and 1 pM the values are below 60%, rising to values above 90% once concentration values reach the 10 pM. This is most likely a result of an increase in light-matter interaction with the rising of peptide concentration. By increasing the amount of scattered light, the method of this document is capable of a better prediction.

Detection accuracy presents a very similar behaviour to the one observed in the Median Probability. Once we reach the 10 pM, the accuracy reaches a value of 1. Although this reflects a poorer performance for the physiological range, the method is still capable of identifying the peptide presence in those concentrations (above chance-level)—see that both median probability and accuracy values are above 50%.

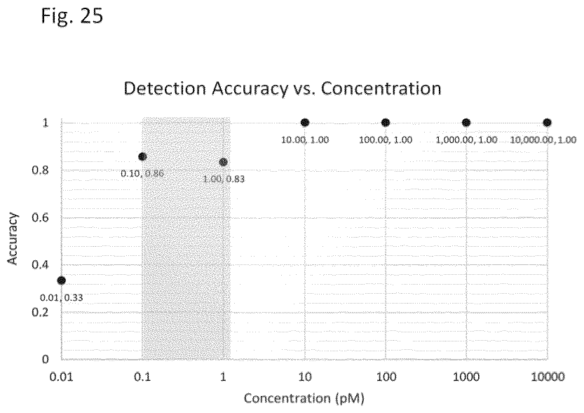

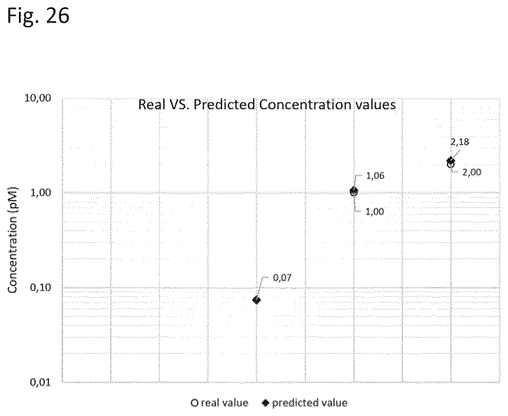

FIGS. 24 and 25 represent, respectively, the ‘Median Probability of Peptide Presence’ and ‘Detection Accuracy’ for peptide concentration (in pM) of the Phosphorylated Tau 441 in diluted human serum. An increase in performance is observed when the concentration of peptide in the serum sample increases. An accuracy above 95% is observed once a 1 pM concentration is reached. This can be explained by an increase in the number of scatter particles that reflect radiation back to the probe, improving the collected scattered signal. For concentrations lower than 1 pM, where the physiological concentration range of the Phosphorylated Tau-441 is included (shaded area in the figure), the performance begins to decrease. Nonetheless, for a concentration of 0.1 pM, an 87% performance is verified, indicating that the technique is still capable of identifying the presence of Phosphorylated Tau-441 at physiological ranges. Unsupervised Learning

Table 5 shows the results of the clustering algorithm for the detection of the Tau 441 peptide. The algorithm could identify two clusters in both datasets. For the total serum samples, the first cluster contains most of the information from the 100 pM, 1000 pM, and 10000 pM samples, while the second encompasses most of the 1 pM samples. The 0 pM, 0.1 pM, and 10 pM samples were randomly distributed between the two clusters. Despite correctly grouping the higher concentration samples, the algorithm was not capable of isolating the samples without peptide.

However, for the 1:2 dilution dataset, the clustering output was different: cluster one gathered 87.5% of all absence samples, while cluster two encompassed most of the samples corresponding to the presence of the peptide. The misclassification rate for this dataset was about 12%, which means that there is a clear distinction between the two types of samples (absence/presence of peptide).

TABLE 5
Percentage of samples belonging to the first cluster from
the two initial projected ones, according to the unsupervised
algorithm for each peptide concentration analysed.

| | Total serum | 1:2 dilution |
|---|---|---|
| Concentration | % within cluster 1 | % within cluster 1 |
| 0 pM | 55.8 | 87.5 |
| 0.1 pM | 60.0 | 10.0 |
| 1 pM | 92.5 | 10.0 |
| 10 pM | 45.0 | 10.0 |
| 100 pM | 0.0 | 20.0 |
| 1000 pM | 2.5 | 20.0 |
| 10000 pM | 2.5 | 0.0 |

### Classification/Distinction of Different Peptides

Table 6 shows the results of the peptide identification task. There was not a significant drop in performance in the test set when comparing to the accuracy in the training set, which means that the models did not overfit. The accuracy was the lowest for the 1 pM samples and increased with the concentration.

The f1-score assumed a similar value to the accuracy indicating that the model is also capable of distinguishing each one of the peptides with the reported performance.

TABLE 6
Performance results for the peptide multiclass identification/classification
task “Amyloid-beta 1-42” vs. “Amyloid-beta 1-
28” vs. “Tau 441” (diluted human serum - 1:2).

| | |
|---|---|
| Train | Test |

| | Accuracy | Accuracy | F1-score |
|---|---|---|---|
| Concentration | (%) | (%) | (%) |
| 1 pM | 76.15 | 70.37 | 69.22 |
| 10 pM | 88.72 | 92.59 | 92.32 |
| 100 pM | 88.97 | 81.48 | 81.86 |
| 1000 pM | 93.46 | 92.59 | 92.56 |

### Peptide Quantification

Table 7 presents the results of the regression analysis to quantify the peptide amount. The algorithm could model the increase in concentration with an r² of 0.98 and an RMSE of 6.03. The discrepancy between the value predicted for the highest concentration and the real value may be explained by the fact that the model was trained with the logarithmic concentration values—in this scale the difference between the value predicted and the actual is minimal. A higher precision could be achieved by training the model with a larger variety of concentrations.

TABLE 7
Regression analysis performance results
(Tau 441, total human serum).

| Concentration | Predicted concentration | | |
|---|---|---|---|
| (pM) | (pM) | r² | RMSE |
| 0.000 | 1.000E-15 | 0.986 | 6.030 |
| 0.100 | 0.345 | | |
| 1.000 | 1.185 | | |
| 10.000 | 11.431 | | |
| 100.000 | 151.143 | | |

### Method of Operation of the Device

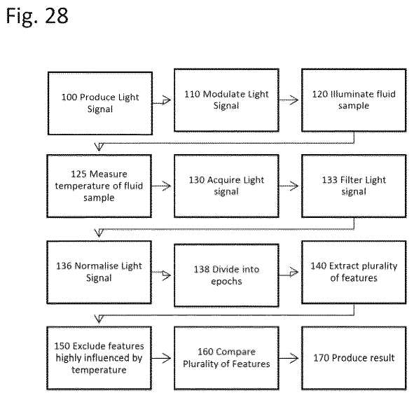

FIG. 28 shows an outline of the method of operation of the device. In a first step 100 a light signal is produced from the laser 1. This light signal is modulated in step 110 as described above before the fluid sample 9 is illuminated in step 120 through a sensing probe 8 with a microlens. In step 130, the light signal from the fluid sample 9 is acquired using the photodetector 7. The light signal is filtered to remove low-frequency components in step 133 and normalized in step 136. The light signal can be divided into periods of time (epochs) in step 138. From this light signal is extracted in step 140 a plurality of features, as outlined above. The features highly influenced by temperature (r>0.70 are excluded from the feature set used in the classification in 150. The updated extracted plurality of features is compared in step 160 with one or more models in a database using the computer 17 and the result of the comparison is output in step 170.

### Experimental Methods

### Peptide Protocol Preparation

Lyophilized 50 μg of the recombinant human Tau-441 (AnaSpec, Fremont, CA, USA, Model #AS-55556-50), liquid 20 μg of the Phosphorylated recombinant human Tau-441 protein (Abcam, Cambridge, UK, Catalog #ab269024), lyophilized 0.5 mg of synthetic Amyloid-beta 1-42 (AnaSpec, Fremont, CA, USA, Model #AS-24224) and Amyloid-beta 1-28 (AnaSpec, Fremont, CA, USA, Model #AS-24231) peptides were prepared following the manufacturer's recommendations. The peptides were thawed at room temperature (RT) before being reconstituted. An aqueous solution of 10 mM NaOH was freshly prepared and filtered (using a 0.02 μm syringe filter) to use as the solvent for the Amyloid-beta 1-42 and Amyloid-beta 1-28 peptides preventing the formation of pre-aggregates. A solution of phosphate-buffered saline (1× PBS) was used to dissolve the Tau-441 peptide.

The Amyloid-beta peptides were initially dissolved by adding 40 μL of 10 mM NaOH, and the Tau-441 by adding 40 μL of 1× PBS to the powder peptide. The phosphorylated form of the Tau-441 was already dissolved. This step was followed by immediate dilution with 1× PBS solution to a concentration of approximately 1 mg/mL or less. The solutions were gently vortexed to mix. The serial peptide concentrations were prepared by diluting the peptides in pooled human serum or in a solution with the same pooled human serum diluted in a ratio of 1:2 in a 1× PBS solution. Each concentration prepared was resuspended several times before use. The remaining stock solution was aliquoted and stored at −80° C.

### Human Serum Protocol Preparation

Human serum pooled gender (BiolVT, Model #HMN320377A, samples #HMN350432

to #HMN350436) processed from whole blood collections was used to do the experiments. The samples were stored at -80° C. and, prior to use, the pooled human serum aliquots were thawed on ice to prepare serial dilutions of the peptides. Peptide dilutions were prepared both in the pooled human serum medium and in a solution of pooled human serum diluted in a ratio of 1:2 in 1× PBS.

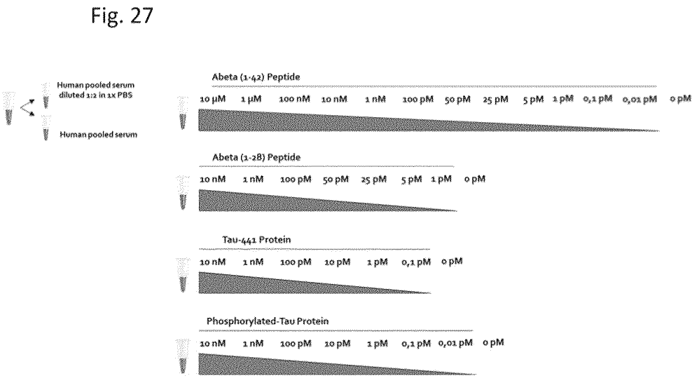

Samples were diluted following the appropriate dilution factor to meet the concentrations of table 1 and according to the scheme of FIG. 27. Additional results

Two types of experiments were conducted to demonstrate the method. The first experiment involved peptides detection, differentiation, and quantification. This first experiment was designed to show the capability of the method and apparatus for detecting peptides in a complex liquid dispersion sample, such as human serum or plasma. The limit of detection in terms of peptide concentration was tested and the ability of the method to identify the spiking of different peptides/proteins in complex media (human serum) at the same concentration. The first experiment also shows the performance in identifying different peptides when present at the same concentration in a complex fluid, and its capability of quantifying the peptides concentration present in the analysed dispersion.

Metabolite detection and quantification. This second experiment was designed to demonstrate the method's capability of detecting metabolites in a complex liquid dispersion sample, such as human serum or plasma, and the corresponding limit of detection in terms of metabolites concentration; and, finally, its capability of quantifying the metabolite concentration present in the analysed dispersion.

### Peptide Detection, Differentiation, and Quantification

The peptide detection, differentiation, and quantification tests were conducted for five peptides/proteins: C-Reactive Protein (CRP), Interleukin-6 (IL-6), Amyloid-beta 1-40 (AB1-40), Galectin-1, and Transthyretin (TTR). CRP and IL-6 are key inflammatory molecules widely associated with acute inflammation as well as severity and progression of chronic conditions, like cancer and COVID-19. Besides the association with cancer, Galectin-1 has several emerging roles in cardiovascular diseases including acute myocardial infarction, heart failure, Chagas cardiomyopathy, pulmonary hypertension, and ischemic stroke. The ratio of Aβ₁₋₄₀/Aβ₁₋₄₂ in blood-derived samples has been shown to predict individual brain amyloid-β-positive or -negative status determined by amyloid-β-PET imaging and used for the diagnosis of Alzheimer's disease.

Previously, it has been reported that the technology detects and quantifies Aβ₁₋₄₂. Here, we explored the detection and quantification of Aβ₁₋₄₀. Lastly, TTR transports the thyroid hormone thyroxine (T4) and retinol-binding protein (RBP) in serum and cerebrospinal fluid. Pathogenic mutations in TTR decrease the stability of their tetramers, enhancing their dissociation into monomers. These monomers can self-aggregate into oligomers and protofibrils that assemble to generate insoluble amyloid fibrils. TTR mutations are therefore involved in several amyloidogenic diseases, such as transthyretin amyloidosis and familiar polyneuropathy.

The peptide detection, differentiation, and quantification tests included spike-in experiments in which the peptides were diluted at predetermined concentrations in relevant biological suspensions. The tested concentrations for the peptides are presented in Table 8 and were determined considering the physiological concentration in human blood. In the particular case of TTR, only differentiation experiments were performed to identify between wild-type (wtTTR) and an amyloidogenic mutated form of TTR (TTR78). For each test, samples with distinct concentrations were analysed from the lowest to the highest concentration, using the same and single probe. Aβ₁₋₄₀ and TTR spike-in samples were prepared in phosphate-buffered saline (PBS); CRP samples in a solution of 4% bovine serum albumin (BSA) diluted in phosphate-buffered saline (PBS), and in foetal bovine serum (FBS); IL-6 and Galectin-1 samples in human serum. CRP detection and quantification was further validated in human serum samples previously analysed using gold-standard laboratory methods. In total, 72 human serum samples were analysed, with a CRP concentration range of 0.3-628 mg/L and an average of 111.7±151.3 mg/L. The average age of the participants was 68±15 years old, and 47% were male. A cleaning procedure (5% bleach followed by water) was applied between samples acquisition to prevent cross-contamination from one sample to the other.

TABLE 8
Peptides' concentrations analysed in
the detection and quantification experiments.

| | |
|---|---|
| CRP concentration | 0 \| 0.0005 \| 0.005 \| 0.05 \| 0.5 \| 1.5 \| |
| (mg/L) | 2.5 \| 5 \| 12.5 \| 15 \| 25 \| 37.5 \| |
| | 50 \| 125 \| 150 \| 250 \| 375 \| 500 |
| IL-6 concentration | 0 \| 0.001 \| 0.01 \| 0.1 \| 1 \| 2.5 \| 5 \| |
| (pg/mL) | 10 \| 25 \| 50 \| 100 \| 1000 \| 10 000 |
| Aβ₁₋₄₀ concentration | 0 \| 1 \| 5 \| 10 \| 35 \| 50 \| 70 \| 100 \| |
| (pg/mL) | 200 \| 350 \| 500 \| 700 \| 1000 |
| Galectin-1 concentration | 0 \| 0.0001 \| 0.001 \| 0.01 \| 0.1 \| 1 \| 5 \| |
| (ng/ml) | 10 \| 25 \| 50 \| 100 \| 1000 \| 10 000 |

### Metabolite Detection and Quantification

Metabolite detection and quantification tests were performed for glucose and insulin, in human samples previously quantified using gold-standard methods. Additionally, a surrogate method was developed to detect urinary creatinine from the analysis of human serum samples (indirect measurement of urinary creatinine). Samples were collected from 56 patients in two independent timepoints (4 months apart), totalling 112 samples for each detection and quantification test. The average age of the participants was 55±8 years old, and 43% were male. Glucose concentration levels in serum samples ranged from 80 mg/dL to 139 mg/dL with an average of 108±12 mg/dL, while insulin concentration varied from 3 μU/mL to 123 μU/mL with an average of 17±16 μU/mL. Creatinine concentration values in urine samples ranged from 352 mg/L and 2924 mg/L, with an average of 1458±554 mg/L.

### Sample Preparation

### Peptide Solutions Preparation Protocol

Lyophilized 1 mg of the native C-reactive protein (Cloud-Clone Corp, Wuhan, China, Catalog #NPA821Hu02), 0.5 mg of the Amyloid-beta 1-40 (AnaSpec, Fremont, CA, USA, Catalog #AS-24235), 5 ug of the Interleukin-6 (PeproTech, Rocky Hill, NJ, USA, Catalog # 200-06) and 10 ug of Galectin-1 (PeproTech, Rocky Hill, NJ, USA, Catalog # 450-39) were prepared following the manufacturer's recommendations. The peptides were thawed or maintained for 15 minutes at room temperature (RT) before being reconstituted. An aqueous solution of 10 mM NaOH was freshly prepared and filtered (using a 0.02 μm syringe filter) to use as the solvent for the Amyloid-beta 1-40 preventing the formation of pre-aggregates. After being initially dissolved, the Aβ₁₋₄₀ was immediately diluted with a solution of phosphate-buffered saline (1× PBS) to a concentration of approximately 1 mg/mL or less. CRP, IL-6, and Galectin-1 were reconstituted in a solution of 1× PBS.

The serial peptide concentrations were prepared by diluting the peptides in the biologically relevant solutions previously mentioned which were further diluted in a ratio of 1:2 in 1× PBS solution for analysis. Each concentration prepared was resuspended several times before use. The remaining stock solution was aliquoted and stored at −80° C.

### Human Serum Preparation Protocol

Human serum pooled gender (BioIVT, Catalog #HMN320377A, samples #HMN350432 to #HMN350436) processed from whole blood collections was used to do the experiments. The samples were stored at −80° C. and, prior to use, the pooled human serum aliquots were thawed on ice to prepare serial dilutions of the peptides. Peptide dilutions were prepared in a solution of pooled human serum diluted in a ratio of 1:2 in 1× PBS.

Human samples used to directly detect and quantify peptides and metabolites were thawed on ice, diluted in a ratio of 1:2 in 1× PBS and analysed. For spike-in experiments, human serum pooled gender samples (BioIVT, Catalog #HMN320377A, samples #HMN350432 to #HMN350436) were thawed on ice prior to the preparation of the serial dilutions with peptides. In all conditions, the pooled serum was kept at a ratio of 1:2 in 1× PBS.

Artificial Intelligence methods for detection and quantification of peptides and metabolites.

### Peptides Detection Among Different Concentration Values

The model was trained to distinguish between the presence and absence of the different peptides in the solutions (binary problem). A distinct model was built to detect each one of the peptides. The “absence class” was composed by acquisition samples of serum without the spiked peptide, whereas the “presence class” was composed of acquisitions samples of serum with the added peptide in different concentrations, depending on which ones of the peptides should be detected. In experiences, where the “absence class” had a smaller number of samples, the “presence class” was randomly under sampled, to build a balanced training set. The model used to perform the classification was the Support Vector Machine (SVM) since the SVM is capable of dealing with either with linear and non-linear input data and the SVM is very suitable for high-dimensionality problems. The SVM can distinguish between two different groups by finding a separating hyperplane with a maximal margin between the classes. Three general attributes define the SVM classifier: C—a hyper-parameter which controls the trade-off between margin maximization and error minimization, the kernel—a function that maps the training data into a high-dimensional feature space and, the sigma, which controls the size of the kernel. Several combinations of these parameters were tested to find the optimal model. Each model was trained using a cross-validation strategy. The optimal model was chosen based on the accuracy across all the validation folders.

### Performance Evaluation

Since each acquisition was divided into epochs and the features calculated from these epochs were fed into the AI model, a prediction was made for each one of the epochs. However, the goal was to evaluate the performance of the model in detecting the presence of the peptide at different concentrations. Thus, three different methods can be considered to calculate this performance.

Epoch accuracy: accuracy of the binary classification considering each epoch for each concentration.

Probability of presence: Median probability of detecting the peptide across all the samples corresponding to the same concentration.

Most frequent performance: Obtained through the plot of the histogram of the predicted detection probabilities across all samples. The performance for each concentration is the bin with the most counts, that corresponds to the most frequently predicted probability range. Peptide Differentiation

A supervised learning pipeline was developed to distinguish between types of peptides. A different model was created to differentiate between each pair of the peptides and the metabolites. The supervised learning algorithms used were support vector machines (SVM) and random forests (RF). The models were trained using a cross-validation. Every model was optimized to find its best parameters, according to the accuracy across the validation folders. Performance evaluation

Each optimized model was tested in the held-out test set (30% of the whole dataset), and its performance was evaluated by computing a complete metrics report. Due to the small number of samples present in the test set, metrics were calculated without epoch grouping, meaning that epochs were considered independent from each other. The report included the area under the receiver operating characteristic curve (AUROC), Accuracy, Precision, and Recall.

### Peptides/Metabolites Detection and Quantification

Regression Analysis

One of the methods used to determine the concentration of the peptides/metabolites was based on the application of supervised learning regressors: Random Forest Regressor and Support Vector Machine. A cross validation strategy was used to train each model. The model performance was evaluated using the r² coefficient, and the best model was chosen according to the evaluation. The training and test samples were divided randomly. The training set encompassed 70% of the samples, while the test set represented 30% of the samples.

### Performance Evaluation

The error in the regressor predictions was measured using the Root Mean Squared Error of the logarithmic concentration values (RMSE), the Mean Absolute Error (MAE) and the r² coefficient.

### Quantification Through Classification by Different Concentration Ranges

One of the alternative methods applied to obtain information about the concentration of the metabolites was based on the application of a supervised learning classifier, the Support Vector Machine. For the CRP, Glucose and Creatinine, the data was split into different classes that represent different concentration ranges. For example, the CRP data was split in two different ways: <100 mg/L vs >=100 mg/L and <=25 mg/L vs >=100 mg/L. In other words, the data was split with a close threshold (100 mg/L) and with a concentration gap between the two classes. Other concentration thresholds were also applied to define new classes for the evaluated peptides (Glucose, Creatinine, and Insulin), based on concentration ranges available.

A cross validation strategy was used to train the model. The training and test samples were divided randomly. The training set encompassed 70% of the samples, while the test set represented 30%. A binary classification approach based on the distinction of ‘low’ versus ‘high’ concentration levels was run for all peptides. An additional multiclass (‘low’ vs ‘medium’ vs ‘high’) classification was applied for Glucose.

### Performance Evaluation

The optimized model was tested in the held-out test set (30% of the whole dataset), and its performance was evaluated by computing a complete metrics report. The performance report included the Accuracy, Precision, Recall and Specificity scores. Particularly for the binary classification, the area under the receiver operating characteristic curve (AUROC) was also calculated.

### Results

Results are divided in the following sections: peptides detection, peptides differentiation, and peptides/metabolites detection and quantification.

### Peptide Detection Among Different Concentration Values

A unique model was developed for the detection of each peptide. The results of each model will be presented separately.

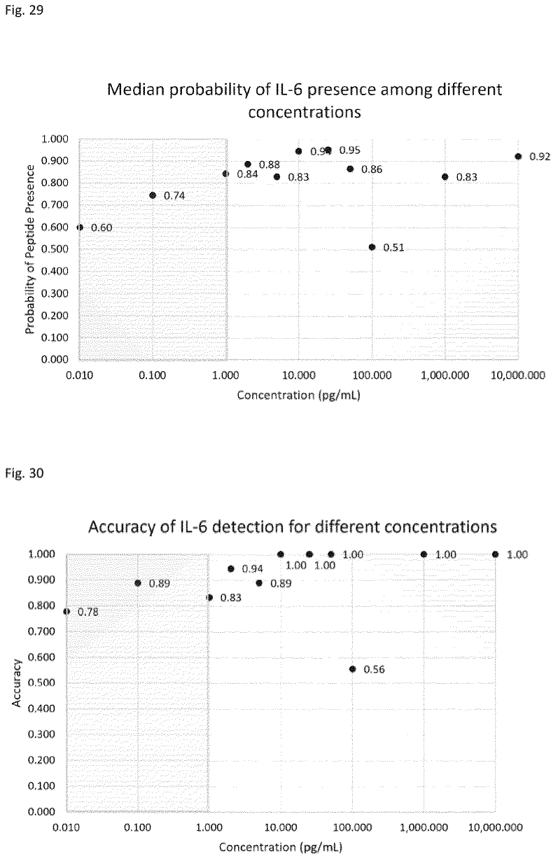

FIG. 29 shows the results for IL6. It is possible to observe the median probability of detecting IL-6 for the different concentrations and FIG. 30 shows the respective detection accuracies. The accuracy is close to 1 for concentrations above 10 pg/mL, except for the solution containing IL-6 at 100 pg/mL. Below that concentration, the accuracy drops slightly, being 78% for the lowest concentration. Therefore, the low performance for the solution at 100 pg/mL is in line with an outlier, most likely caused by external factors to the acquisition. Despite the decrease in predictions confidence the model can classify concentrations in the biological range accurately.

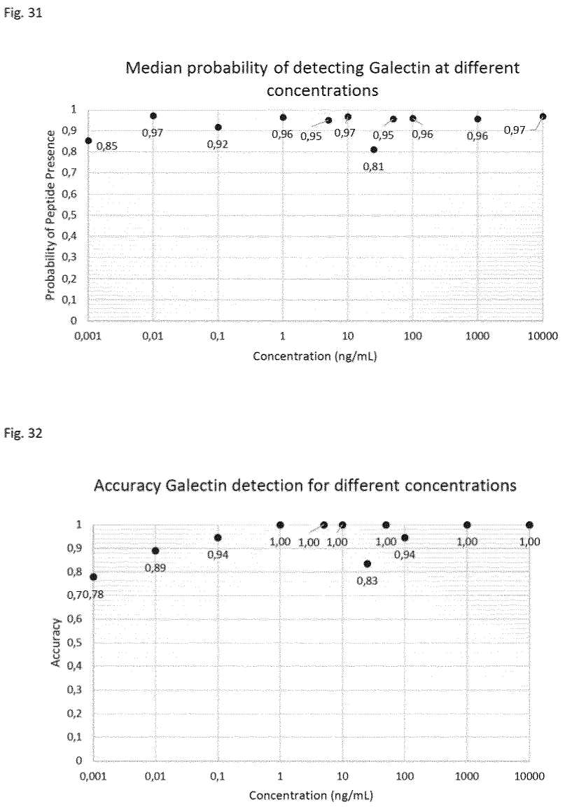

The probability of detecting Galectin in solutions that contains Galectin is higher than 80% independently of the concentration, meaning that the classifier is confident in its predictions (see FIG. 31). FIG. 32 shows that the detection accuracy is close to 1 for concentrations above 1 ng/ml and has a small drop for values below that. The performance in the biological range is above 80%. The smallest accuracy, 78%, is registered for the 0.001 ng/ml solution. The behaviour of the classifier follows the intuition behind the problem: the peptide is harder to detect when present in small concentrations. Nevertheless, the model can do it with performance highly above chance.

### Peptide Differentiation

A distinct model was built for the various peptide's differentiation tasks. The method can be used to differentiate peptides with an accuracy above 90%. Hereafter, the results for the tested classification tasks will be presented and discussed.

The results of the differentiation between the wild-type TTR (wtTTR) and the mutated TTR78 are presented in Table 9. As observed, the SVM model achieved values above 90% regarding all the performance metrics.

TABLE 9
Performance of Transthyretin differentiation in the held-out test set.

| Classification | Test | Test | Test | |
|---|---|---|---|---|
| Task | Accuracy | Precision | Recall | AUROC |
| Class 1: wtTTR | 92.86% | 94.74% | 90.00% | 93.02% |
| Class 2: TTR78 | | | | |

The results of the differentiation between Galectin and IL-6 in the held-out test set are presented in Table 10. All metrics are close to 100%, showing that the model can confidently distinguish between the two peptides.

TABLE 10
Metrics report for the differentiation between IL-6 and Galectin.

| Classification | Test | Test | Test | |
|---|---|---|---|---|
| Task | Accuracy | Precision | Recall | AUROC |
| Class 1: Galectin. | 98.71% | 98.32% | 99.15% | 99.71% |
| Class 2: IL-6 | | | | |

### Peptides/Metabolites Detection and Quantification

The results of the different quantification tasks are presented below. A different model was developed for each different metabolite/peptide. The methodologies used for the quantification varied: a regression model was used for amyloid-beta 1-40, IL-6 and Galectin, whereas quantification based on concentration ranges was used for the CRP, Glucose, Creatinine, and Insulin.

A unique model was developed for each one of the different peptides: C Reactive Protein (CRP), Amyloid-beta 1-40, IL-6, and Galectin.

C Reactive Protein (CRP) As observed in Table 11, the model obtained good performance overall. The performance is higher when there is a concentration gap between the two classes, and the task proved to be easier in FBS than in plasma.

TABLE 11
CRP concentration level classification performance - Random Forest classifier.

| | Classification | Test | Test | Test | Test | Test |
|---|---|---|---|---|---|---|
| Matrix/Medium | Problem | F1-score | Precision | Recall | Specificity | AUROC |
| FBS | Close threshold | 0.752 | 0.827 | 0.689 | 0.856 | 0.850 |
| | Class 1: Conc. <100 mg/L; | | | | | |
| | Class 2: Conc. ≥100 mg/L | | | | | |
| | Gap threshold | 0.811 | 0.682 | 1.000 | 0.725 | 0.989 |
| | Class 1: Conc. ≤25 mg/L; | | | | | |
| | Class 2: Conc. ≥100 mg/L | | | | | |
| Plasma | Close threshold | 0.685 | 0.704 | 0.667 | 0.667 | 0.667 |
| | Class 1: Conc. <100 mg/L; | | | | | |
| | Class 2: Conc. ≥100 mg/L | | | | | |
| | Gap threshold | 0.734 | 0.836 | 0.654 | 0.792 | 0.803 |
| | Class 1: Conc. ≤25 mg/L; | | | | | |
| | Class 2: Conc. ≥100 mg/L | | | | | |

Amyloid-beta 1-40. The performance of the regression model for the quantification of amyloid-beta 1-40 in the held-out test set is shown in Table 12. The r² coefficient is 0.65, meaning that the model can approximate the predictions to the real data points. Although the model can discriminate a relationship between the concentrations of the solutions, it does not do it very accurately since the MAE is high. Table 12 depicts the predictions made by the model, the trendline that fits them (dashed line), and the desired relationship (dotted line, light blue).

TABLE 12
Amyloid-beta 1-40 quantification performance.

| | Test | Test | Test |
|---|---|---|---|
| Peptide | r² | RMSE | MAE |
| Amyloid-beta 1-40 | 0.653 | 222.20 | 175.24 |
| (0-1000 pg/mL) | | | |

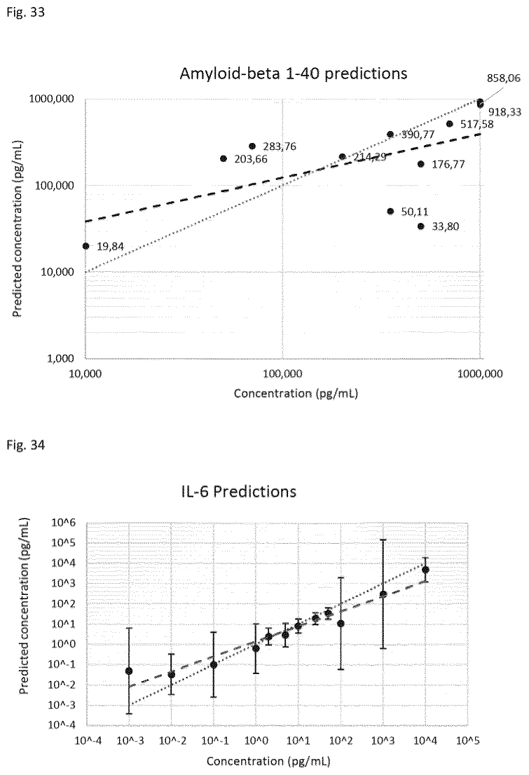

FIG. 33 shows predictions made by Amyloid-beta quantification model. The dashed line represents the trendline that fits the points, while the dotted line depicts the desired relationship.

Table 13 shows the metrics report for the performance of the IL-6 quantification model in the held-out test set. The r² coefficient is 0.93, indicating that the model can accurately explain the inputs. It can then effectively model the relationship between the optical fingerprint and the peptide concentration. The low values of the RMSE and MAE corroborate this hypothesis. Table 19 shows the model predictions and the corresponding error bars, the trendline that fits them, and the ideal line constructed with the perfect predictions. The fitted line is close to the ideal one, since the errors are small. However, the error bars are larger for the small concentrations, showing that the quantification is harder for those values.

TABLE 13
IL-6 quantification model performance in the test set.

| | Test | Test | Test |
|---|---|---|---|
| Peptide | r² | RMSE | MAE |
| IL-6 | 0.935 | 1.427 | 0.954 |
| (0-10000 pg/mL) | | | |

FIG. 34 shows predictions made by IL-6 quantification model and respective error bars. The dashed line represents the trendline that fits the points, while the dotted line depicts the desired relationship.

Galectin. Table 14 presents the complete metrics report for the results of the galectin quantification in the test set. The prediction errors are small—both the RMSE and MAE are below one. The r² coefficient is 0.97, showing that the model can successfully quantify the peptide. Based on FIG. 34, it is possible to conclude that the trendline fitted on the model's predictions approximates almost the ideal scenario.

TABLE 14
Metrics report for the galectin quantification
model performance in the test set

| | Test | Test | Test |
|---|---|---|---|
| Peptide | r² | RMSE | MAE |
| Galectin | 0.973 | 0.988 | 0.819 |
| (0-10000 ng/ml) | | | |

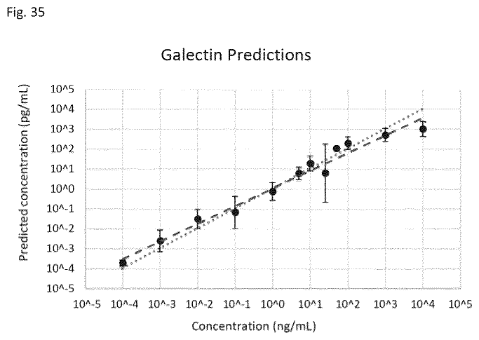

FIG. 35 shows predictions made by galectin quantification model and respective error bars. The dashed line represents the trendline that fits the points, while the dotted line depicts the desired relationship.

Metabolites. The results for the quantification of metabolites using concentration ranges are presented hereafter. A different model was developed for the different task: quantification of Glucose, Urinary Creatinine, and Insulin. The second was an indirect measurement.

Glucose As observed in Table 15, the model achieved very good performance for the gap threshold and even for the close threshold, with the area under the ROC curve always above 80%.

TABLE 15
Glucose concentration level classification performance.

| | Test results | | | | |
|---|---|---|---|---|---|
| Classification Problem | F1-score | Precision | Recall | Specificity | AUROC |
| Close threshold | | | | | |
| Class 1: Conc. <110 mg/dL | 0.744 | 0.718 | 0.771 | 0.721 | 0.828 |
| Class 2: Conc. >=110 mg/dL | | | | | |
| Gap threshold | | | | | |
| Class 1: Conc. <=100 mg/dL | 0.911 | 0.926 | 0.897 | 0.928 | 0.969 |
| Class 2: Conc. >=120 mg/dL | | | | | |
| Gap threshold | | | | | |
| Class 1: Conc. <=90 mg/dL | 0.921 | 0.875 | 0.972 | 0.861 | 0.972 |
| Class 2: Conc. >=130 mg/dL | | | | | |

For the specific case of the glucose, a multiclass classification model achieved a very satisfactory performance and shows a good potential to achieve a regression algorithm in the future (Table 16).

TABLE 16
Glucose concentration level multiclass classification.
Glucose

| | Test results | | | | |
|---|---|---|---|---|---|
| Classification Problem | Accuracy | F1-score | Precision | Sensitivity | Specificity |
| Class 1: Conc. <=100 mg/dL | 0.675 | 0.676 | 0.681 | 0.676 | 0.838 |
| Class 2: Conc. >100 and <120 mg/dL | | | | | |
| Class 3: Conc. >=120 mg/dL | | | | | |

### Urinary Creatinine.

The performance of the classifier for the urinary creatinine regarding the area under the ROC curve is always above 80%. The performance increases when the gap between the two classes increases, as expected.

TABLE 17
Creatinine concentration level classification performance.

| | Test results | | | | |
|---|---|---|---|---|---|
| Classification Problem | F1-score | Precision | Sensitivity | Specificity | AUROC |
| Close threshold | | | | | |
| Class 1: Conc. <1500 mg/L | 0.763 | 0.748 | 0.778 | 0.725 | 0.818 |
| Class 2: Conc. >=1500 mg/L | | | | | |
| Gap threshold | | | | | |
| Class 1: Conc. <=1200 mg/L | 0.706 | 0.779 | 0.645 | 0.808 | 0.804 |
| Class 2: Conc. >=1800 mg/L | | | | | |
| Gap threshold | | | | | |
| Class 1: Conc. <=1000 mg/L | 0.759 | 0.732 | 0.789 | 0.708 | 0.846 |
| Class 2: Conc. >=2000 mg/L | | | | | |
| Gap threshold | | | | | |
| Class 1: Conc. <=800 mg/L | 0.887 | 0.859 | 0.917 | 0.845 | 0.963 |
| Class 2: Conc. >=2200 mg/L | | | | | |

### Insulin

The performance of the classifier for the insulin was above 75% for the close threshold (first row) with regards to AUROC and above 80% for the gap threshold classification problem as observed in table 18.

TABLE 18
Insulin concentration level classification performance.

| | Test results | | | | |
|---|---|---|---|---|---|
| Classification Problem | F1-score | Precision | Sensitivity | Specificity | AUROC |
| Close threshold | | | | | |
| Class 1: Conc. <15 μU/mL | 0.680 | 0.704 | 0.658 | 0.733 | 0.787 |
| Class 2: Conc. >=15 μU/mL | | | | | |
| Gap threshold | | | | | |
| Class 1: Conc. <=10 μU/mL | 0.852 | 0.855 | 0.848 | 0.855 | 0.904 |
| Class 2: Conc. >=20 μU/mL | | | | | |

### REFERENCES

[1] Goedert, M., Alzheimer's and Parkinson's diseases: The prion concept in relation to assembled Aβ, tau, and α-synuclein. Science 2015, 349(6248): 1255555. doi: 10.1126/science.1255555
[2] Chatterjee, S. K., Zetter, B. R., Cancer biomarkers: knowing the present and predicting the future. Future Oncol. 2005, 1(1):37-50. doi: 10.1517/14796694.1.1.37
[3] Dhingra, R. and Vasan, R. S., Biomarkers in cardiovascular disease: Statistical assessment and section on key novel heart failure biomarkers. Trends Cardiovasc Med. 2017, 27(2): 123-133. doi: 10.1016/j.tcm.2016.07.005
[4] Wang, J., Tan, G. J. et al., Novel biomarkers for cardiovascular risk prediction. J Geriatr Cardiol. 2017, 14(2): 135-150. doi: 10.11909/j.issn. 1671-5411.2017.02.008
[5] Alcolea, D., Pegueroles, J., et al. Agreement of amyloid PET and CSF biomarkers for Alzheimer's disease on Lumipulse. Ann Clin Transl Neurol. 2019, 6(9): 1815-1824. doi:10.1002/acn3.50873
[6] Lewczuk, P., Matzen, A., et al. Cerebrospinal Fluid Aβ42/40 Corresponds Better than Aβ42 to Amyloid PET in Alzheimer's Disease. J Alzheimers Dis. 2017, 55(2): 813-822. doi:10.3233/JAD-160722
[7] Fossati, S., Ramos Cejudo, J., et al. Plasma tau complements CSF tau and P-tau in the diagnosis of Alzheimer's disease. Alzheimers Dement (Amst) 2019, 11:483-492. doi: 10.1016/j.dadm.2019.05.001
[8] Mattsson, N., Zetterberg, H., et al. Plasma tau in Alzheimer disease. Neurology. 2016, 87(17): 1827-1835. doi:10.1212/WNL.0000000000003246
[9] Risacher, S. L., Fandos, N., et al. Plasma amyloid beta levels are associated with cerebral amyloid and tau deposition. Alzheimers Dement (Amst). 2019, 11: 510-519. doi: 10.1016/j.dadm.2019.05.007
[10] Tzen, K. Y., Yang, S. Y., et al. Plasma Aβ but not tau is related to brain PiB retention in early Alzheimer's disease. ACS Chem Neurosci. 2014, 5(9): 830-836. doi: 10.1021/cn500101j
[11] Song, F., Poljak, A. et al., Meta-Analysis of Plasma Amyloid-β levels in Alzheimer's Disease. Alzheimers Dis. 2011, 26(2): 365-375. doi: 10.3233/JAD-2011-101977
[12] Yang, S., Chiu, M., et al. Analytical performance of reagent for assaying tau protein in human plasma and feasibility study screening neurodegenerative diseases. Sci Rep 2017, 7, 9304. https://doi.org/10.1038/s41598-017-09009-3
[13] Chang, C., Yang, S., Plasma and Serum Alpha-Synuclein as a Biomarker of Diagnosis in Patients with Parkinson's Disease. Frontiers in Neurology 2020, 10. Doi:10.3389/fneur.2019.01388
[14] Ding, J., Zhang, J., et al. Relationship between the plasma levels of neurodegenerative proteins and motor subtypes of Parkinson's disease. J Neural Transm 2017, 124: 353-360. https://doi.org/10.1007/s00702-016-1650-2
[15] Lee, P. H., Lee, G., et al. The plasma alpha-synuclein levels in patients with Parkinson's disease and multiple system atrophy. J Neural Transm (Vienna) 2006, 113(10): 1435-1439. doi: 10.1007/s00702-005-0427-9
[16] Lin, C., Yang, S., et al. Plasma α-synuclein predicts cognitive decline in Parkinson's disease. Journal of Neurology, Neurosurgery & Psychiatry 2017, 88:818-824.
[17] Veerabhadrappa, B., Delaby, C., et al., Detection of amyloid beta peptides in body fluids for the diagnosis of Alzheimer's disease: Where do we stand? Crit Rev Clin Lab Sci. 2020, 57(2): 99-113. doi:10.1080/10408363.2019.1678011
[18] Höglund, K., Wiklund, O., et al., Plasma Levels of β-Amyloid (1-40). β-Amyloid(1-42), and Total β-Amyloid Remain Unaffected in Adult Patients with Hypercholesterolemia After Treatment with Statins. Arch Neurol. 2004, 61(3): 333-337. doi: 10.1001/archneur.61.3.333
[19] Palmqvist, S., Janelidze, S., et al. Performance of Fully Automated Plasma Assays as Screening Tests for Alzheimer Disease-Related β-Amyloid Status. JAMA Neurol. 2019, 76(9): 1060-1069. doi: 10.1001/jamaneurol.2019.1632
[20] Pannee, J., Portelius, E., et al. A Selected Reaction Monitoring (SRM)-Based Method for Absolute Quantification of Aβ 38. Aβ 40, and β 42 in Cerebrospinal Fluid of Alzheimer's Disease Patients and Healthy Controls. Journal of Alzheimer's Disease 2013, 1021 - 1032. doi 10.3233/JAD-2012-121471
[22] Verberk, I. M. W., Slot, R.E., et al. Plasma Amyloid as Prescreener for the Earliest Alzheimer Pathological Changes. Ann Neurol. 2018;84(5):648-658. doi: 10.1002/ana. 25334
[23] Janelidze, S., Stomrud, E., et al. Plasma β-amyloid in Alzheimer's disease and vascular disease. Sci Rep. 2016, 6: 26801. Published 2016 May 31. doi: 10.1038/srep26801
[23] Perez-Grijalba, V., Fandos, N., et al. Validation of Immunoassay-Based Tools for the Comprehensive Quantification of Aβ40 and Aβ42 Peptides in Plasma. J Alzheimers Dis. 2016, 54(2): 751-762. doi:10.3233/JAD-160325
[24] Lue, L., Kuo, Y. and Sabbagh, M. Advance in Plasma AD Core Biomarker Development: Current Findings from Immunomagnetic Reduction-Based SQUID Technology. Neurol Ther 2019, 8: 95-111. https://doi.org/10.1007/s40120-019-00167-2
[25] O. Soppera, C. Turck, D. J. Lougnot, D. Fabrication of micro-optical devices by self-guiding photopolymerization in the near IR. Optics Letters. 2009, 34(4): 461-463. https://doi.org/10.1364/OL.34.000461
[26] O. Soppera, S. Jradi, D. J. Lougnot. Photopolymerization with microscale resolution: Influence of the physico-chemical and photonic parameters. Journal of Polymer Science. 2008, 46: 3783-3794.
   https://doi.org/10.1002/pola.22727
[27] R. S. R. Ribeiro, R. Queirós, O. Soppera, A. Guerreiro, P. A. S. Jorge. Optical fibre tweezers fabricated by guided wave photo-polymerization. Photonics. 2015, 2: 634-645.
   https://doi.org/10.3390/photonics2020634
[28] K. Neuman and S. Block. Optical trapping. Review of Scientific Instruments, 75(9):2787-2809, 2004.
[29] Hart P C, Rajab I M, Alebraheem M, Potempa L A. C-Reactive Protein and Cancer-Diagnostic and Therapeutic Insights. Front Immunol. 2020; 11:595835. Published 2020 Nov. 19. doi: 10.3389/fimmu.2020.595835
[30] Kumari N, Dwarakanath B S, Das A, Bhatt A N. Role of interleukin-6 in cancer progression and therapeutic resistance. Tumour Biol. 2016;37(9):11553-11572. doi:10.1007/s13277-016-5098-7
[31] Liu F, Li L, Xu M, et al. Prognostic value of interleukin-6, C-reactive protein, and procalcitonin in patients with COVID-19. J Clin Virol. 2020; 127:104370. doi:10.1016/j.jcv.2020.104370
[32] Orozco C A, Martinez-Bosch N, Guerrero P E, et al. Targeting galectin-1 inhibits pancreatic cancer progression by modulating tumour-stroma crosstalk. Proc Natl Acad Sci U S A. 2018;115(16):E3769-E3778. doi: 10.1073/pnas. 1722434115
[33] Seropian I M, González G E, Maller S M, Berrocal D H, Abbate A, Rabinovich G A. Galectin-1 as an Emerging Mediator of Cardiovascular Inflammation: Mechanisms and Therapeutic Opportunities. Mediators Inflamm. 2018;2018:8696543. Published 2018 Nov. 5. doi:10.1155/2018/8696543
[34] Nakamura A, Kaneko N, Villemagne V L, et al. High-performance plasma amyloid-β biomarkers for Alzheimer's disease. Nature. 2018;554(7691):249-254. doi: 10.1038/nature25456
[35] Connors L H, Lim A, Prokaeva T, Roskens V A, Costello C E. Tabulation of human transthyretin (TTR) variants, 2003. Amyloid. 2003:10(3): 160-184. doi: 10.3109/13506120308998998
[36] Yee A W, Aldeghi M, Blakeley M P, et al. A molecular mechanism for transthyretin amyloidogenesis. Nat Commun. 2019;10(1):925. Published 2019 Feb. 25. doi: 10.1038/s41467-019-08609-z

### REFERENCE NUMERALS

1 Laser
2 Laser driver
3 Data acquisition board
4 Optical coupler
5 Photodetector
8 Sensing Probe
9 Sample
10 Thermometer
12 Light source
13 Objective
14 Mirror
15 Zoom lens
16 Digital camera
17 Computer

1. A method for identification of amino acid residues in a fluid sample (9) comprising:
producing (100) a light signal from a laser (1);
illuminating (120) the fluid sample (9) with the light signal through a lens in a sensing probe (8);
acquiring (130) a light signal from the fluid sample (9);
extracting (140) a plurality of features from the light signal; and
comparing (150) the extracted plurality of features with a model in a database to determine the amino acid residues in the fluid sample (9).

2. The method of claim 1, further comprising filtering (133) the acquired light signal to remove noisy low-frequency components.

3. The method of any of the above claimsclaim 1, further comprising normalizing (136) the light signal.

4. The method of claim 1, further comprising modulating (110) the light signal from the laser (1).

5. The method of claim 1, wherein the extraction (138) of the plurality of features in the light signal is carried out over periods of time.

6. The method of claim 1, wherein the plurality of features are time domain and frequency derived features.

7. The method of claim 1, further comprising measurement (125) of the temperature of the fluid sample (9).

8. The method of claim 1, wherein the model is created by one of a support vector machine or a clustering algorithm.

9. A device for identification of amino acid residues in a fluid sample (9) comprising:
a laser (1) connected through an optical fiber with a sensing probe (8) with a microlens for illuminating the sample (9);
a detector (16) for acquiring (130) a light signal from the sample (9); and
a computer (17) adapted to analyze the light signal, extract (140) features from the light signal, compare (150) the extracted features with stored features in a database and produce (160) a result.

10. The device of claim 9, further comprising a micromanipulator for manipulating the fluid sample (9).

11. The device of claim 9, wherein the sensing probe (8) comprises a microlens at the end of the optical fiber.

12. The device of claim 9, further comprising a thermometer for measuring (125) the temperature of the sample (9).

13. Use of the method of claim 1 for the detection of neurodegenerative disease, such as Alzheimer's disease, cardiovascular diseases and cancer.

14. A method for creation of a model for identification of amino acid residues in a fluid sample (9) comprising:
producing (100) a light signal from a laser (1);
illuminating (120) a series of fluid samples (9) with known concentrations of the amino acid residues with the light signal through a microlens in a sensing probe (8);
acquiring (130) a light signal from the fluid sample (9);
extracting (140) a plurality of features from the light signal; and
applying a learning method to the extracted plurality of features to correlate the features with the fluid samples (9) to create the model in a database.

15. The method of claim 14, wherein the learning method is at least one of a supervised learning methods, clustering algorithms, or a regression models.

16. The device of claim 10, wherein the sensing probe (8) comprises a microlens at the end of the optical fiber.

17. The device of claim 10, further comprising a thermometer for measuring (125) the temperature of the sample (9).

18. The device of claim 11, further comprising a thermometer for measuring (125) the temperature of the sample (9).

19. The device of claim 16, further comprising a thermometer for measuring (125) the temperature of the sample (9).
